(19) United States
(12) Patent Application Publication
(10) Pub. No.: US 2025/0163498 A1
(43) Pub. Date: May 22, 2025

(54) METHODS AND COMPOSITIONS RELATED TO NUCLEIC ACID-BASED FLUORESCENT NANOCLUSTER PROBES

(71) Applicant: BOARD OF REGENTS, THE UNIVERSITY OF TEXAS SYSTEM, Austin, TX (US)

(72) Inventors: Hsin-Chih Yeh, Austin, TX (US); Soonwoo HONG, Austin, TX (US); Yuan-I CHEN, Austin, TX (US); Yu-An KUO, Austin, TX (US); Trung Duc NGUYEN, Austin, TX (US)

(21) Appl. No.: 18/726,884

(22) PCT Filed: Jan. 4, 2023
(86) PCT No.: PCT/US2023/060100

§ 371 Date: Jul. 5, 2024

Related U.S. Application Data
(60) Provisional application No. 63/296,213, filed on Jan. 4, 2022.
(60) Provisional application No. 63/400,097, filed on Aug. 23, 2022.

(51) Int. Cl. C12Q 1/6823; C12N 9/22; C12N 15/11; C12Q 1/44; C12Q 1/6876; G01N 21/64

(52) U.S. Cl. CPC C12Q 1/6823; C12N 9/22; C12N 15/11; C12Q 1/44; C12Q 1/6876; G01N 21/6428; C12N 2310/20; G01N 2021/6439; G01N 2333/922

(57) ABSTRACT

Disclosed are compositions and methods for detecting nucleic acid, or detecting the presence or absence of a nuclease. Specifically, disclosed herein are nucleic acid-metal nanocluster probes. which comprise a nucleic acid probe with metal nanoclusters associated therewith. These nucleic acid-metal nanocluster probes can be used to detect a variety of cleavage events. and can also be used to detect hybridization to the probe.

### GOVERNMENT SUPPORT CLAUSE

This invention was made with government support under grant number CHE1611451 awarded by the National Science Foundation (NSF). The government has certain rights in the invention.

### CROSS-REFERENCE TO RELATED APPLICATIONS

This application claims benefit of U.S. Provisional Application No. 63/296,213, filed Jan. 4, 2022, and U.S. Provisional Application No. 63/400,097, filed Aug. 23, 2022, both of which are hereby incorporated herein by reference in their entirety.

### BACKGROUND

Since 2016, CRISPR-Cas12a (Chen 2018), -Cas13a (East-Seletsky 2016, Gootenberg 2017), and-Cas13b (Gootenberg 2018) have been found to possess interesting collateral cleavage activities (also called trans nuclease activities) that can be harnessed for nucleic acid detection, resulting in development of two new assays—SHERLOCK (Gootenberg 2017) and DETECTR (Chen 2018) (FIG. 1A). In DETECTR, the collateral cleavage activities by Cas12a enzyme, for example, cut single-stranded DNA (ssDNA, which is a fluorescent probe that we often call “the reporter”) non-specifically upon activation with a single-stranded DNA (ssDNA) or double-stranded DNA (dsDNA) target. In SHERLOCK, the collateral cleavage activities by Cas13 enzymes (Cas13a and Cas13b) cut single-stranded RNA reporter non-specifically upon activation with an ssRNA target. The reporters used in the current CRISPR/Cas assays (SHERLOCK and DETECTR) are dual-labeled FRET probes whose fluorescence is originally quenched in the reaction mix but recovered after cleavage by the activated Cas nucleases (FIG. 1A). Thus, the enhanced fluorescence signal indicates the existence of viral nucleic acids in the sample. CRISPR/Cas assays have shown a number of advantages over the CDC gold standard RT-qPCR assay in SARS-CoV-2 detection: (1) they use an isothermal amplification process such as RT-LAMP (Broughton 2020) or RT-RPA (Chen 2018; Gootenberg 2017; Gootenberg 2018; Kaminski 2020: Ackerman 2020) to boost the viral RNA, thus bypassing the need to use an expensive thermal cycler at a central laboratory for detection (i.e., equipment-free); (2) the cleaved reporters (which are the end products of the assays) can be easily read by a properly designed paper strip, making them promising assays for COVID-19 diagnosis at POC settings (Joung 2020) (i.e., user-friendly); and (3) they can reliably detect SARS-CoV-2 viral RNA in 40-60 mins (including manual extraction of viral RNA from a nasopharyngeal swab) (Broughton 2020), as compared to 4-6 hours in RT-qPCR assay (Sheridan 2020) (i.e., rapid and robust). In terms of the specificity, well-designed CRISPR/Cas assays can discriminate targets at the single-nucleotide resolution, similar to the specificity of RT-qPCR assay. However, in terms of the sensitivity, the current CRISPR/Cas assays have the limit of detection (LOD) 10 times worse than that of the RT-qPCR assay (10 viral copies vs. 1 viral copy per μl in fluorescence readout (Broughton 2020). Whereas the unbeatable sensitivity of PCR makes PCR remain the gold standard assay for infectious disease diagnostics at the CDC, PCR can only be run at central laboratories. In contrast, CRISPR/Cas assays can become an easy assay to run at home with sensitivity and specificity approaching to those of PCR

One key component in the current CRISPR/Cas assays is the dual-labeled FRET probe that allows for the fluorescence detection of the target nucleic acids. FRET probes, such as molecular beacons (Tyagi and Kramer, 1996) and Taqman probes (Gelfand 1991), are often used in fluorescence assays. These FRET probes have their donor's fluorescence quenched by a nearby quencher moiety in their original forms, making these probes dark. Upon configurational change or digestion of the FRET probe molecules, the donor's fluorescence is recovered, making these probes fluoresce.

Although FRET probes have enabled many sensitive and highly specific assays, FRET probes have a number of limitations, particularly in the field of detection of nucleic acids and nuclease activities. They are expensive to design and manufacture, and they are difficult to multiplex. They still cost ˜$300 for 1 nanomole of ssRNA-based or 6.5 nanomole ssDNA-based FRET reporters. Besides, most FRET probes only allow for single-channel emission measurement (single color) due to their intrinsic donor-quencher system, which also causes relatively high background signal. There exists in the art a need for improved probes for detecting nucleic acid cleavage events rapidly, sensitively, reliably and quantitatively. Ideal probes would give rise to minimal background signal and be easily and inexpensively prepared.

Fluorescent metal nanoclusters, such as DNA-based silver nanoclusters (DNA-AgNCs), are emerging fluorescence tools in imaging, catalysis, sensing, and biomedicine. DNA-AgNCs display superior optical performance since their size is close to the Fermi wavelength. DNA-AgNCs possess unique features, including high fluorescence quantum yields and stability, biocompatibility, facile synthesis, and low toxicity, which are requisite for fluorescent probes. The fluorescent emission of DNA-AgNCs can cover the violet (UV, 300 nm) to near-infrared (NIR, 950 nm) region by varying the DNA sequences, lengths, and structures or by modifying the environmental factors (such as buffer, pH, metal ions, macromolecular polymers, and small molecules) (Yang M, Chen X, Su Y, Liu H, Zhang H, Li X and Xu W (2020) The Fluorescent Palette of DNA-Templated Silver Nanoclusters for Biological Applications. Front. Chem. 8:601621).

What is needed in the art are non-FRET-based reporter molecules, such as the fluorescent metal nanoclusters disclosed herein, which are capable of rapidly and inexpensively detecting digestion of nucleic acids.

### SUMMARY

Disclosed herein is a composition comprising: (a) a probe which can indicate the presence of a target nucleic acid, the probe comprising a nucleic acid sequence and a fluorescent metal nanocluster associated with the nucleic acid sequence; and (b) a nuclease capable of cleaving the nucleic acid sequence of the probe.

Also disclosed is a composition comprising: (a) a probe which can indicate the presence of a target nucleic acid, the probe comprising a nucleic acid sequence and a fluorescent metal nanocluster associated with the nucleic acid sequence; and (b) a buffer, wherein said buffer comprises a buffer that stabilizes the fluorescent metal nanocluster, the associated nucleic acid sequence and the nuclease.

Further disclosed is a probe which can indicate the presence of a target nucleic acid, the probe comprising: a nucleic acid sequence, wherein said nucleic acid sequence comprises a nuclease recognition site, and a fluorescent metal nanocluster associated with the nucleic acid sequence.

Disclosed is a probe which can indicate the presence of a target nucleic acid, the probe comprising: a nucleic acid sequence, wherein said nucleic acid sequence is about 3-150 nucleotides in length, and a fluorescent metal nanocluster associated with the nucleic acid sequence.

Also disclosed is a probe which can indicate the presence of a target nucleic acid, the probe comprising: a nucleic acid sequence, wherein said nucleic acid sequence can further comprise a secondary structure, and a fluorescent metal nanocluster associated with the nucleic acid sequence.

Further disclosed is a composition comprising at least one probe, wherein one probe can indicate the presence of at least two different target nucleic acids; wherein the probe comprises a nucleic acid sequence and a fluorescent metal nanocluster associated with the nucleic acid sequence; wherein the probe fluoresces differently upon binding of different target nucleic acids.

Disclosed is a composition comprising at least two probes, wherein each probe can indicate the presence of a different target nucleic acid; wherein each probe comprises a nucleic acid sequence and a fluorescent metal nanocluster associated with the nucleic acid sequence; wherein each probe fluoresces differently upon binding of different target nucleic acids.

Also disclosed is a method of detecting a target nucleic acid in a sample, the method comprising (a) contacting the sample with: (i) at least one ribonucleoprotein (RNP) comprising a guide molecule specific for at least one target nucleic acid and an effector molecule which is capable of cleaving a probe upon binding of the guide molecule in the presence of target nucleic acids; and (ii) at least one probe comprising a nucleic acid sequence and a fluorescent metal nanocluster associated with the nucleic acid sequence; and (b) measuring a detectable signal produced by cleavage of the probe by the effector molecule upon binding of the guide molecule, thereby detecting the target nucleic acid.

Further disclosed is a method of detecting nuclease activities, the method comprising: (a) contacting the sample with: (i) a nuclease which is capable of cleaving a probe upon binding of the guide molecule; and (ii) at least one probe comprising a nucleic acid sequence and a fluorescent metal nanocluster associated with the nucleic acid sequence; and (b) measuring a detectable signal produced by cleavage of the probe by the nuclease, thereby detecting the target nucleic acid.

Disclosed is a method of detecting the presence of multiple target nucleic acids in a single reaction, the method comprising: (a) contacting the sample with a probe, wherein said probe comprises: (i) at least two nucleic acid sequences which are capable of hybridizing with two or more target nucleic acids; (ii) at least one fluorescent metal nanocluster associated with the nucleic acid sequence; and (c) measuring a detectable signal, wherein hybridization of one probe gives a first signal, hybridization of a second probe gives a second signal, and hybridization of both probes gives a third signal; thereby detecting the presence of multiple target nucleic acids.

### DESCRIPTION OF DRAWINGS

FIG. 1A-C shows a schematic diagram of CRSIPR-associated diagnostics using collateral cleavage activities: DETECTR and SHERLOCK. Upon activation by target DNA/RNA, they can collaterally cut the reporter probe showing fluorescence changes. (B) The probe based on DNA/AgNCs can be light-up upon digestion, and (C) novel reporters for CRISPR/Cas assays can be universally utilized.

FIG. 2A-F shows the digestion by DNase I generates different pieces of fragment of which showed fluorescence changes. The changes in color or intensity were dramatic with DNA/AgNCs-based reporters. (B-E) Our reporters showed the more significant changes after loading DNase I than (F) DNaseAlert. Depending on the template sequence, they showed different types of fluorescence changes: (B) turn-on, (C) turn-off, and (D, E) color change.

FIG. 3A-B shows in-gel validation of FAM-labeled DNA/AgNC probes upon digestion. Two different DNA/AgNCs were used: (A) with a 10-nt template, AAT CCC CCC A (SEQ ID NO: 4); (B) with a 26-nt template, GGG TTA GGG TCC CCC CAC CCT TAC CC (SEQ ID NO: 2). The raw reporters both showed originally two bands (*, **), but the top band (*) disappeared (or got dimmer) with DNase I and Cas12a enzymes. The third and fourth bands (***) appeared upon digestion only with the activated Cas12a (A) or DNase I (B), showing there was a clear digestion on DNA/AgNC probes.

FIG. 4 shows three different ways the DNA/AgNC probes can be used: with a nuclease, with a CRISPR/Cas system, and with multiple probes.

FIG. 5 shows DNA-templated silver nanoclusters (DNA/AgNCs) can be used to create single-stranded RNA or DNA probes.

FIG. 6 shows a method of replacing the currently used dual-labeled FRET probes in CRISPR/Cas assays, and generating cleavage barcodes.

FIG. 7 shows the design of “cleavage barcodes” and multiplexed Cas detection using only one Cas and one universal reporter.

FIG. 8 shows three samples that were tested with DNase I which clearly show results of cleavage.

FIG. 9 shows fluorescence changes and fluorescent fragments after cleavages in: hRP1-1 (44 nucleotides: TCCCCAAC AA TTGTC CGACCTGCAGTGAT GACAA AA TCCCCAAC, SEQ ID NO: 5), hRP2-1 (40 nucleotides, TCCCCAAC TTGTC CGACCTGCAGTGAT GACAA TCCCCAAC, SEQ ID NO: 6) and hRP3 (55 nucleotides, CCCTTAATCCCC AA TTGTC CGACCTGCAGTGAT GACAA AA CCCCCTAATTCCCCC SEQ ID NO: 7). DNase I clearly cut DNA regardless of the presence of AgNCs. DNase I didn't work without the buffer. (Lane 17-18). 495LP filter used; 23% polyacrylamide gel ran at75V for 400 minutes; 20 μM for DNA/AgNCs and DNA loaded; 2 μM for DNaseAlert loaded. Buffer (purchased from NEB)-1X composition: 10 mM Tris-HCl 2.5 mM MgCl₂ 0.5 mM CaCl₂ pH 7.6 at 25°.

FIG. 10A-B shows hairpin structure for DNA templates. A shows a schematic, B shows an example.

FIG. 11 schematic for identifying DNA/AgNCs candidates using polyacrylamide gel electrophoresis (PAGE).

FIG. 12 shows UV excitation results (ISO 500, 1/40 s, UV shield applied)

FIG. 13 shows that the individual bands can interact with DNase I/Cas12a in a different way. Gel results 2 (17-32, AGT . . . G to GAG . . . G) 25% polyacrylamide gel, 45V for 20 hours, 180 μM DNA/AgNCs.

FIG. 14 shows that a high concentration of magnesium acetate (Mg(OAc)₂) inhibits DNase I digestion, but up to 10 mM still allows it to function.

FIG. 15 shows Cas12a is compatible with ammonium acetate+magnesium acetate or sodium phosphate buffer with magnesium acetate. (Mg ions are key component for Cas12a activation).

FIG. 16 shows an illustration, where fluorescent noble metal nanoclusters are collections of small numbers of gold or silver atoms (2-30 atoms).

FIG. 17 shows binding of AgNO₃ and/or NaBH₄ to ssDNA, which acts as a ligand. Sequence-dependent emissive properties are then detected.

FIG. 18 shows the concept of digestion-and-turn-on probes to replace dual-labeled probes.

FIG. 19 shows real-time fluorescence changes of Subak probe under 365 nm UV excitation. While the working condition for DNase I activation is at 37° C., the DNase I digestion can be demonstrated by the heat generated by a UV transilluminator.

FIG. 20 shows that Digest-on is a non-FRET based probe that change its color upon nuclease digestion. Gel-purification can improve the yield rate of probes.

FIG. 21 shows “digest-on” probes can be used with CRISPR/Cas12a to detect target nucleic acids.

FIG. 22 shows that the Subak probe can be used in colorimetric assay as well as fluorescence assay.

FIG. 23A-B shows mass spectrometry revealed three major fragments in the stem of the hairpin DNA template.

FIG. 24 shows other similar design probes support the cleavage site on the stem.

FIG. 25 shows that the color change occurs because the total number of silver atoms increases from Ag13 to Ag14.

FIG. 26A-B shows the probes disclosed herein can detect the presence of a target nucleic acid in combination of CRISPR/Cas such as Cas12a with the same fluorescence change (100 nm red-shift). (A) is the control, (B) is Cas12a with and without target.

FIG. 27A-B shows hairpin-designed DNA/AgNCs as cleavable probes. Depicted are SEQ ID NOS: 9 and 10 (SEQ ID NO: 9 is the hairpin and the hRP20-12, SEQ ID NO: 10 is hRP41-46).

FIG. 28A-C shows a summary of variant samples before and after digestion. FIG. 28A summarizes the fluorescence of variants before and after digestion. Three heat maps are seen; each one shows the relative intensity compared to that of the original Subak probe (hRP20-12). X axis represents the original base and position (for example C3 indicates a cytosine at the 3rd from the 5′ end (hRP20-12). And Y axis indicates the base change in each mutation. So, A at C3 means that cytosine was replaced at the 3rd, and this change makes it 2.5 times in green emission and 1.1 times in red emission before and after digestion, respectively. FIG. 28B shows an example of fluorescence of the raw, with buffer, and with DNase I. FIG. 28C shows hRP41-46 (T12G) highlighted with a blue box. The cousins of this hRP41-46 have similar enhancement: hRP41-44, hRP41-45.

### DETAILED DESCRIPTION

### General Definitions

Unless defined otherwise, all technical and scientific terms used herein have the same meaning as commonly understood to one of ordinary skill in the art to which this disclosure belongs.

Ranges can be expressed herein as from “about” one particular value, and/or to “about” another particular value. By “about” is meant within 10% of the value, e.g., within 9, 8, 8, 7, 6, 5, 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. Throughout the description and claims of this specification the word “comprise” and other forms of the word, such as “comprising” and “comprises,” means including but not limited to, and is not intended to exclude, for example, other additives, components, integers, or steps.

As used in the specification and claims, the singular form “a”, “an”, and “the” include plural references unless the context clearly dictates otherwise. For example, the term “an agent” includes a plurality of agents, including mixtures thereof.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce,” or “abrogate,” (used interchangeably) or other forms of the word, such as “reducing” or “reduction,” or “abrogating” or “abrogation” is meant lowering of an event or characteristic. It is understood that this is typically in relation to some standard or expected value, in other words it is relative, but that it is not always necessary for the standard or relative value to be referred to.

By “increase” or other forms of the word, such as “increasing,” is meant raising or elevating. It is understood that this is typically in relation to some standard or expected value, in other words it is relative, but that it is not always necessary for the standard or relative value to be referred to.

As used herein, by a “subject” is meant an individual. Thus, the “subject” can include domesticated animals (e.g., cats, dogs, etc.), livestock (e.g., cattle, horses, pigs, chickens, ducks, geese, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician.

“Control” refers to a sample or standard used for comparison with an experimental sample. In some embodiments, the control is a sample obtained from a healthy subject (or a plurality of healthy subjects), such as a subject or subjects not expected or known to have a particular polymorphism. In additional embodiments, the control is a historical control or standard reference value or range of values (such as a previously tested control sample or plurality of such samples), or group of samples that represent baseline or normal values. A positive control can be an established standard that is indicative of a specific methylated nucleotide. In some embodiments a control nucleic acid is one that lacks a particular methylated nucleotide, and is used in assays for comparison with a test nucleic acid, to determine if the test nucleic acid includes the methylated nucleotide.

“Detecting” is used herein to identify the existence, presence, or fact of something. General methods of detecting are known to the skilled artisan and may be supplemented with the protocols and reagents disclosed herein. For example, included herein are methods of detecting a nucleic acid molecule in sample. Detection can include a physical readout, such as fluorescence output.

“Enhancer Sequence” refers to a nucleotide sequence that when placed in proximity to another nucleic acid molecule having templated metal nanoclusters increases the fluorescence intensity of the metal nanocluster when exposed to excitation light. Exemplary enhancer sequences are known in the art and disclosed herein.

“Excitation Light” refers to light of any wavelength that is capable of causing template metal nanoclusters to fluoresce. Non-limiting examples of excitation light include visible light, ultraviolet and near infrared light.

The term “hybridization” is defined as forming base pairs between complementary regions of two strands of DNA, RNA, or between DNA and RNA, thereby forming a duplex molecule, for example. Hybridization conditions resulting in particular degrees of stringency will vary depending upon the nature of the hybridization method and the composition and length of the hybridizing nucleic acid sequences. Generally, the temperature of hybridization and the ionic strength (such as the Na+ concentration) of the hybridization buffer will determine the stringency of hybridization. Calculations regarding hybridization conditions for attaining particular degrees of stringency are discussed in Sambrook et al., (1989) Molecular Cloning, second edition, Cold Spring Harbor Laboratory, Plainview, N.Y. (chapters 9 and 11).

An “isolated” biological component (such as a nucleic acid molecule) has been substantially separated, produced apart from, or purified away from other biological components. Nucleic acid molecules which have been “isolated” include nucleic acids molecules purified by standard purification methods, as well as those chemically synthesized. Isolated does not require absolute purity, and can include nucleic acid molecules that are at least 50% isolated, such as at least 75%, 80%, 90%, 95%, 98%, 99% or even 100% isolated.

A “nucleation sequence” is a sequence of nucleotides capable of binding or associating with metal atoms to form template metal nanoclusters. The portion of a nucleic acid molecule including a nucleation sequence of nucleotides is referred to as the “nucleation portion” of the nucleic acid molecule. Exemplary nucleation sequences are known and provided herein. Specific nucleation sequences that are useful for interacting with metal nanoclusters and forming DNA templated metal nanoclusters are disclosed herein. Examples of metal nanoclusters for use as fluorescent reporters, and methods of producing templated metal nanoclusters on DNA oligonucleotides are known. See, e.g., U.S. Patent Publication No. 20110212540, incorporated by reference herein in its entirety, and U.S. Publication No. 20140349289, incorporated herein by reference.

A “nucleic acid” is a deoxyribonucleotide or ribonucleotide polymer, which can include analogues of natural nucleotides that hybridize to nucleic acid molecules in a manner similar to naturally occurring nucleotides. In a particular example, a nucleic acid molecule is a single stranded (ss) DNA or RNA molecule, such as a probe or primer. In another particular example, a nucleic acid molecule is a double stranded (ds) nucleic acid, such as a target nucleic acid. Examples of modified nucleic acids are those with altered backbones, such as peptide nucleic acids (PNA).

The major nucleotides of DNA are deoxyadenosine 5′-triphosphate (dATP or A), deoxyguanosine 5′-triphosphate (dGTP or G), deoxycytidine 5′-triphosphate (dCTP or C) and deoxythymidine 5′-triphosphate (dTTP or T). The major nucleotides of RNA are adenosine 5′-triphosphate (ATP or A), guanosine 5′-triphosphate (GTP or G), cytidine 5′-triphosphate (CTP or C) and uridine 5′-triphosphate (UTP or U).

Nucleotides include those nucleotides containing modified bases, modified sugar moieties and modified phosphate backbones, as known in the art.

Examples of modified base moieties which can be used to modify nucleotides at any position on its structure include, but are not limited to: 5-fluorouracil, 5-bromouracil, 5-chlorouracil, 5-iodouracil, hypoxanthine, xanthine, acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N-6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N-2-carboxypropyl)uracil, and 2,6-diaminopurine.

Examples of modified sugar moieties which may be used to modify nucleotides at any position on its structure include, but are not limited to: arabinose, 2-fluoroarabinose, xylose, and hexose, or a modified component of the phosphate backbone, such as phosphorothioate, a phosphorodithioate, a phosphoramidothioate, a phosphoramidate, a phosphordiamidate, a methylphosphonate, an alkyl phosphotriester, or a formacetal or analog thereof.

The term “complementary binding” as used herein occurs when the base of one nucleic acid molecule forms a hydrogen bond to the base of another nucleic acid molecule. Normally, the base adenine (A) is complementary to thymidine (T) and uracil (U), while cytosine (C) is complementary to guanine (G). For example, the sequence 5′-ATCG-3′ of one ssDNA molecule can bond to 3′-TAGC-5′ of another ssDNA to form a dsDNA. In this example, the sequence 5′-ATCG-3′ is the reverse complement of 3′-TAGC-5′. Nucleic acid molecules can be complementary to each other even without complete hydrogen-bonding of all bases of each molecule. For example, hybridization with a complementary nucleic acid sequence can occur under conditions of differing stringency in which a complement will bind at some but not all nucleotide positions.

A “polymorphism” is a variation in a gene sequence. The polymorphisms can be those variations (DNA sequence differences, e.g., substitutions, deletions, or insertions) which are generally found between individuals or different ethnic groups and geographic locations which, while having a different sequence, produce functionally equivalent gene products. Typically, the term can also refer to variants in the sequence which can lead to gene products that are not functionally equivalent. Polymorphisms also encompass variations which can be classified as alleles and/or mutations which can produce gene products which may have an altered function. Polymorphisms also encompass variations which can be classified as alleles and/or mutations which either produce no gene product or an inactive gene product or an active gene product produced at an abnormal rate or in an inappropriate tissue or in response to an inappropriate stimulus. Alleles are the alternate forms that occur at the polymorphism.

Polymorphisms can be referred to, for instance, by the nucleotide position at which the variation exists, by the change in amino acid sequence caused by the nucleotide variation, or by a change in some other characteristic of the nucleic acid molecule or protein that is linked to the variation.

“Probes,” as used herein, refer to short nucleic acid molecules, usually DNA or RNA oligonucleotides, typically of about 3-150 nucleotides in length, and more specifically, about 6-100 nucleotides in length, used to detect the presence of a complementary target nucleic acid strand in a sample. All or a portion of a probe can be annealed to a complementary target nucleic acid strand by nucleic acid hybridization to form a hybrid between the probe and the target DNA strand. The probes are associated with metal nanoclusters. Therefore, nucleic acid-metal nanocluster probes can be used to identify a target nucleic acid molecule, wherein the sequence of the probe is specific for the target nucleic acid molecule, for example so that the probe will hybridize to the target nucleic acid molecule under very high stringency hybridization conditions.

Typically, the nucleic acid portion of the probe includes at least about 6 contiguous nucleotides, such as at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or about 50 contiguous nucleotides, that are complementary to a target nucleic acid molecule, such as 20-70 nucleotides, 20-60 nucleotides, 20-50 nucleotides, 20-40 nucleotides, or 20-30 nucleotides. Probes can also be of a maximum length, for example no more than 20, 25, 25, 40, 50, 75 or 100 nucleotides in length. The specificity of a particular probe typically increases with an increase in the number of complementary nucleotides on the probe.

The probe can also include additional nucleotides that are not complementary to the target nucleic acid molecule. The additional nucleotides can be used, for example, for detection of the probe in a sample. In several embodiments, the probes disclosed herein include a hybridization portion that is complementary to a test nucleic acid sequence, and a nucleation portion (that can associate with metal nanoclusters) or an enhancer portion (that can enhance the fluorescence of metal nanoclusters associated with the nucleation portion). The additional nucleotides can be located 5′ or 3′ of the hybridization nucleotides.

Methods for preparing and using nucleic acid probes are described, for example, in Sambrook et al. (In Molecular Cloning: A Laboratory Manual, CSHL, New York, 1989), Ausubel et al. (ed.) (In Current Protocols in Molecular Biology, John Wiley & Sons, New York, 1998), and Innis et al. (PCR Protocols, A Guide to Methods and Applications, Academic Press, Inc., San Diego, Calif., 1990).

The identity/similarity between two or more nucleic acid sequences is expressed in terms of the identity between the sequences. Sequence identity can be measured in terms of percentage identity; the higher the percentage, the more identical the sequences are.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biotechnology (NCBI, National Library of Medicine, Building 38A, Room 8N805, Bethesda, Md. 20894) and on the Internet, for use in connection with the sequence analysis programs blastp, blastn, blastx, tblastn, and tblastx. Additional information can be found at the NCBI web site. BLASTN is used to compare nucleic acid sequences. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1554 nucleotides is 75.0 percent identical to the test sequence (1166-1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. In another example, a target sequence containing a 20-nucleotide region that aligns with 20 consecutive nucleotides from an identified sequence as follows contains a region that shares 75 percent sequence identity to that identified sequence (that is, 15-20*100=75). One indication that two nucleic acid molecules are closely related is that the two molecules hybridize to each other under stringent conditions, as described above.

A “sample,” such as a biological sample, is a sample obtained from a subject. As used herein, biological samples include all clinical samples useful for detection of a methylated nucleotide, including, but not limited to, cells, tissues, and bodily fluids, such as: blood; derivatives and fractions of blood, such as serum; urine; sputum; or CVS samples. In a particular example, a sample includes blood obtained from a human subject, such as whole blood or serum.

A “test nucleic acid molecule” refers to a nucleic acid molecule whose detection, quantitation, qualitative detection, characterization, or a combination thereof, is intended. For example, the test nucleic acid molecule can be a defined region or particular portion of a nucleic acid molecule, for example a portion of a genome (such as a gene or a region of DNA or RNA containing a gene or portion thereof of interest). The nucleic acid molecule need not be in a purified form. Various other nucleic acid molecules can also be present with the test nucleic acid molecule. For example, the test nucleic acid molecule can be a specific nucleic acid molecule (which can include RNA or DNA), for which the detection of a particular polymorphism is intended. In some examples, a test nucleic acid includes a viral nucleic acid molecule, or a bacterial nucleic acid molecule. Purification or isolation of the test nucleic acid molecule, if needed, can be conducted by methods known to those in the art, such as by using a commercially available purification kit or the like.

By “contacting” is meant placement in direct physical association, for example solid, liquid or gaseous forms. Contacting includes, for example, direct physical association of fully-and partially-solvated molecules.

A “metal nanocluster” is a collection of small numbers (e.g., 2-30 atoms) of noble metal atoms (e.g., gold or silver atoms) with physical sizes close to the Fermi wavelength of an electron (\tilde{ }3 nm for gold and silver). The metal ions can have affinity for nitrogen atoms on DNA, including the N3 of cytosine and the N7 of guanine. Metal nanoclusters for use with the disclosed embodiments are fluorescent, that is, they have the ability to emit light of a particular wavelength (emission wavelength) when exposed to light of another wavelength (excitation wavelength). Specific nucleotide sequence (“nucleation sequences”) that are useful for interacting with metal nanoclusters and forming DNA templated metal nanoclusters are disclosed herein. Examples of metal nanoclusters for use as fluorescent reporters, and methods of producing templated metal nanoclusters on DNA oligonucleotides are known. See, e.g., U.S. Pat. App. Pub. 2011/0212540 entitled “Probe and Method for DNA Detection”, which was filed Feb. 22, 2011, and is incorporated by reference herein in its entirety. See also Richie et al., “Ag Nanocluster Formation using a cytosine oligonucleotide template,” J Phys Chem C, 111, 175-181, 2006, which is incorporated by reference herein in its entirety.

### Nucleic Acid-Metal Nanocluster Probes

Disclosed herein is a probe which can indicate the presence of a target nucleic acid, the probe comprising a nucleic acid sequence and a fluorescent metal nanocluster associated with the nucleic acid sequence. In other words, the probe comprises two portions; a metal nanocluster and a nucleic acid portion.

Nucleic acid-metal nanoclusters are multicolor fluorophores associated with a nucleic acid whose emission fluorescence can be tuned by rearranging the nucleobases surrounding the clusters. Whereas introducing extra nucleobases to the vicinity of a metal nanocluster can significantly enhance its fluorescence, it has never been shown that removing nucleobases around a metal nanocluster can also enhance its fluorescence. The encapsulated metal nanocluster lights up significantly with a shift in the emission peaks. Although the digestion by DNase I and Cas12a (for example) is a nonspecific cleavage, a digestion pattern can be seen when the template is stained with a silver nanocluster. The digestion pattern was validated using gel electrophoresis showing two specific digestion sites in the middle of the template sequence (Example 1). The results not only show that nucleic acid-metal nanocluster probes can be a nuclease activity sensor, but also provide insights into the interactions between nucleases and metallo-nanoclusters.

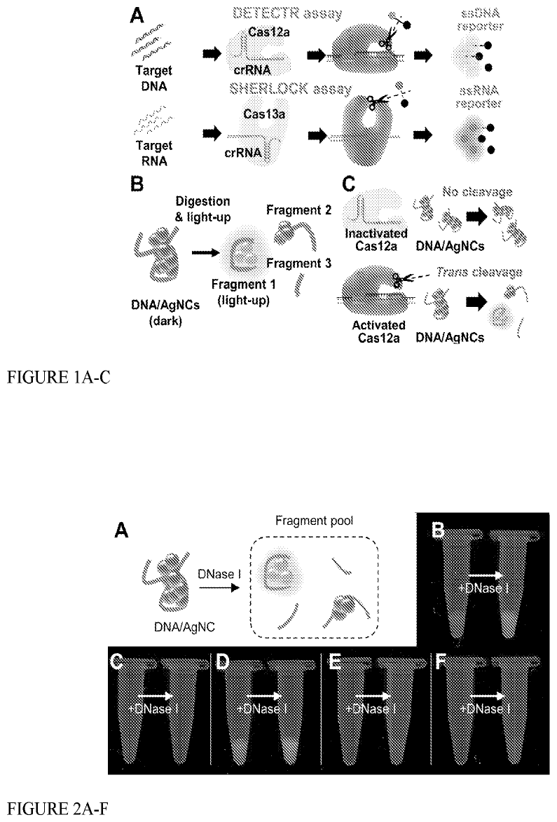

The nucleic acid-metal nanocluster probes disclosed herein are highly fluorescent (with quantum yields up to 0.9 and extinction coefficients ˜105 M⁻¹ cm⁻¹) (Petty 2018; Obliosca 2013; Neascu 2020), multicolor (emission peaks ranging from 300-950 nm) (Obliosca 2013; Choi 2012), activatable (Yeh 2010; Obliosca 2014; Petty 2013), color tunable (Petty 2014; Del Bonis-O'Donnell 2019; Cerretani 2019), environmentally sensitive (Obliosca 2013; Choi 2012), and biocompatible Sharma 2012). DNA or RNA can template the growth of few-atom silver nanoclusters (Ag₈, Ag₁₀ or Ag₁₆) (Huard 2018; Petty 2016; Cerretani 2019), creating a palette of organic-inorganic composite nanomaterial fluorophores (FIG. 1B). Nucleic acid-metal nanocluster probes such as DNA/AgNCs are unique probes, as the photophysical properties of metal nanoclusters, such as silver clusters can reflect subtle changes in their surrounding ligand (i.e., nucleobase) environment, making them superior sensors for a number of applications (Obliosca 2013), including DNA (Obliosca 2014; Sharma 2010), SNP (Yeh 2012), DNA methylation (Chen 2015), and enzyme activity (Juul 2015) detection. The nucleic acid-metal nanocluster probes disclosed herein are alternatively referred to as “Digest-on” probes or “Subak probes.”

While a large fluorescence enhancement was achieved upon “contact” interactions with a guanine base (Yeh 2011; Obliosca 2013), it has never been reported that their fluorescence changes upon digestion (FIG. 1B, 1C). Not only is a this a new, noble reporter for digestion-based sensors, but it also improves the sensitivity of current CRISPR/Cas assays by 1, 2, 3, 4, 5, 6, 7, 8, 9, or 10-fold or more, preferably 5-fold or more, which approaches the sensitivity of RT-qPCR, while maintaining the native advantages of the CRISPR/Cas assays. A collateral benefit of this approach is that the new reporters created for the CRISPR/Cas assays are about 100-fold cheaper than the TaqMan reporter used in the current qPCR assay.

Multiple probes can be used simultaneously. For example, two or more probes can be used that have different metal nanocluster components, or different nucleic acid components, or both. These different probes can have different properties, such that differential detection of a target, or quantification of a target, is possible. This is discussed in more detail below.

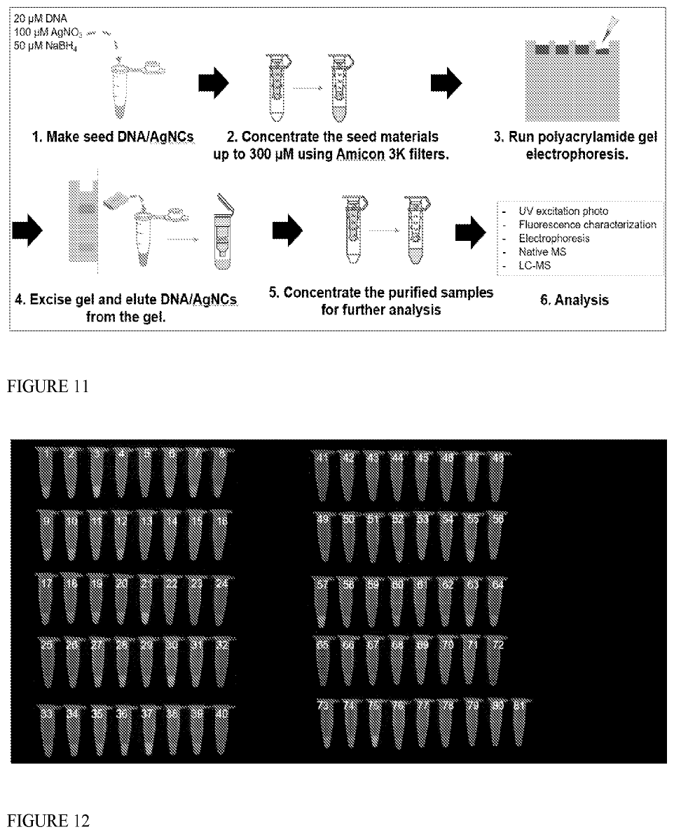

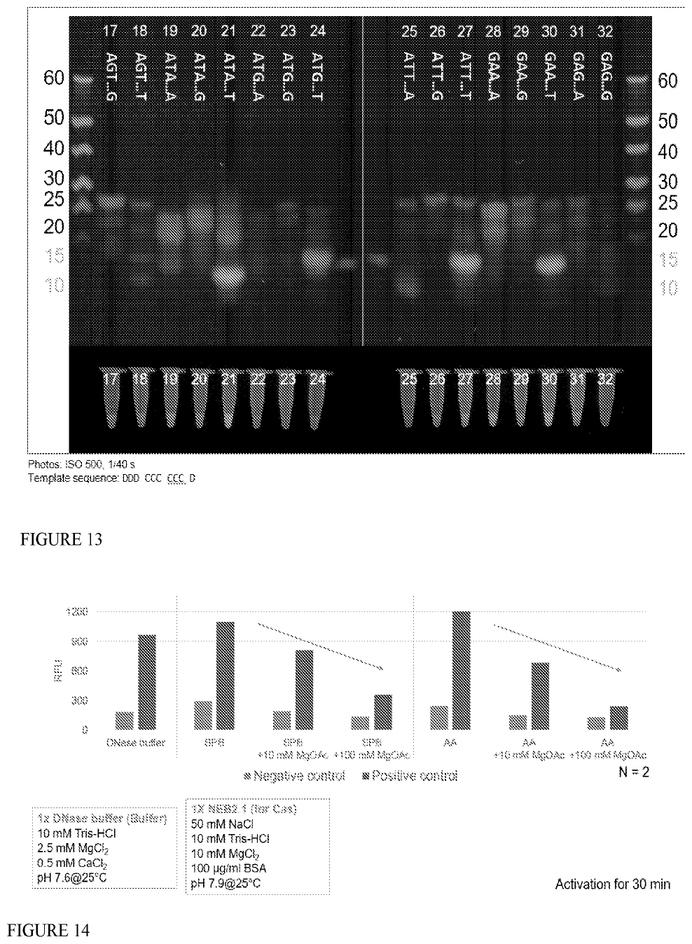

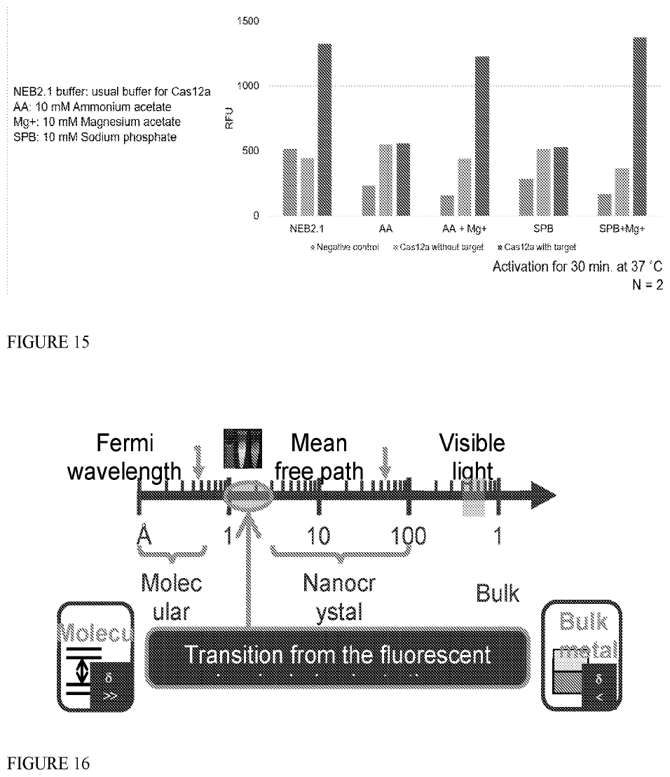

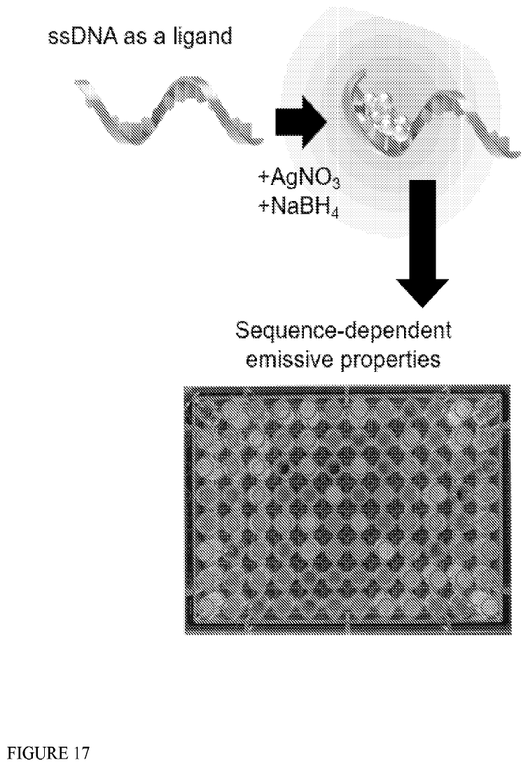

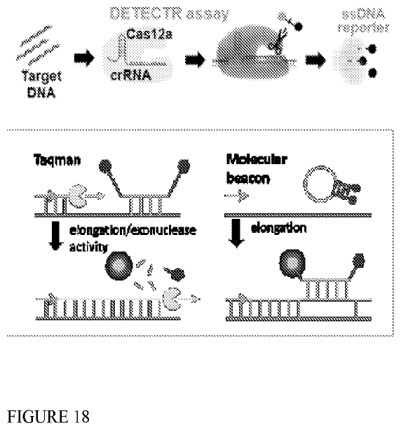

The nucleic acid-metal nanoclusters disclosed herein have two main components: the nucleic acid probe portion, and the metal nanoclusters associated therewith. Each of these are discussed in detail below. An illustration of the nucleic acid-metal nanocluster can be seen in FIG. 11A.

### Metal Nanocluster Portion

The disclosed embodiments take advantage of the differential fluorescent properties of metal (e.g., silver) nanoclusters when the nanoclusters are brought near different nucleic acid sequences. The metal of the templated metal nanoclusters can be a noble metal, such as silver, gold, or copper. Particularly envisioned herein is a silver nanocluster (AgNC). Nucleic acid-templated silver nanoclusters can emit colored light. The color of the emitted light was found to depend on the particular nucleic acid sequence it is associated with. Importantly, this emitted light can shift when different nucleic acids are brought in contact with the AgNC. Silver nanoclusters are groups of from about 2 to about 30 silver atoms that are about less than 3 nm in size with the properties of good fluorescence, good photostability, and electroluminescence. These silver nanoclusters can function as fluorescence reporters of nucleic acid digestion or differential hybridization.

To form metal nanoclusters on DNA, positively charged metal ions (e.g., Ag⁺ atoms) are first attached to ssDNA spontaneously in solution. Then, a reductant (e.g., sodium borohydride) is added to reduce the charge of the atoms (e.g., Ag⁺ to Ag (0)), after which metal atom “clusters” will form. The ssDNA prevents the metal cluster “from growing out of control”. Clusters that become a “nanoparticle” (size>5 nm) are not fluorescent.

Examples of metal nanoclusters for use as fluorescent reporters, and methods of producing templated metal nanoclusters on DNA oligonucleotides are known. See, e.g., U.S. Pat. App. Pub. 2011/0212540 entitled “Probe and Method for DNA Detection”, which was filed Feb. 22, 2011, and is incorporated by reference herein in its entirety. See also Richie et al., “Ag Nanocluster Formation using a cytosine oligonucleotide template,” J Phys Chem C, 111, 175-181, 2006, which is incorporated by reference herein in its entirety. The basis for the operation of the templated metal nanoclusters is a controlled conversion of DNA-templated silver nanoclusters between a dark, non-emissive state, which is their state when not associated with an enhancer sequence, and a bright, emissive state when associated with the enhancer sequence. Unlike prior use of metal nanoclusters, the present method involves tuning the fluorescent emission properties of the metal nanoclusters (e.g., a wavelength shift of 60-70 nm) by altering the relative positions of the nanoclusters to the and the enhancer sequence.

The disclosure of the following references and their description of templated metal nanoclusters and their use and detection is incorporated by reference herein in its entirety: Petty et al., J American Chemical Society 2004, 126, 5207; Vosch et al., Proc. Natl. Acad. Sci. U.S.A. 2007, 104, 12616; Gwinn et al., Adv. Mater. 2008, 20, 279; Petty et al., Anal. Chem. 2011, 83, 5957; Sharma et al., Chem. Commun. 2010, 46, 3280; Sharma et al., Chem. Commun. 2011, 47, 2294; Neidig et al., J. Am. Chem. Soc. 2011, 133, 11837; and Yeh et al., Nano Lett. 2010, 10, 3106.

Noble metal nanoclusters, such as those made of silver, gold, copper, or other noble metals typically include collections of a number of metal atoms (approximately 2-30 atoms or 2, 3, 4, 5, 6, 7, 8, 9, 10, 11, 12, 13, 14, 15, 16, 17, 18, 19, 20, 21, 22, 23, 24, 25, 26, 27, 28, 29 or 30 atoms) with physical sizes close to the Fermi wavelength of an electron (e.g., about 0.5 nm for gold and silver). They behave like molecular systems and yield fluorescence emission in the UV-visible and infrared range. In some examples, oligonucleotide-templated silver nanoclusters (“DNA/Ag NCs”), which are a versatile set of fluorophores that have been used for a variety of applications including live cell imaging, detection of specific metal ions, and single-nucleotide variation identification. DNA/Ag NCs can be biocompatible and can have better photostability than commonly used organic dyes. Unlike organic dyes and photoluminescent nanocrystals, they are subject to silver oxidation/reduction or nanocluster (“NC”) regrouping, which results in conversion among different NC species. These different species may provide different color emissions.

Also contemplated herein are composite nanoclusters made of more than one material, such as silver and copper.

Detection of fluorescence emission can be performed according to known methods, for example as described herein. The excitation light can be selected from the group consisting of ultraviolet light, visible light, near infrared light or a combination thereof. In a related aspect, the wavelength of excitation light is from 200 nm to 2000 nm (or 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1050, 1100, 1150, 1200, 1250, 1300, 1350, 1400, 1450, 1500, 1550, 1600, 1650, 1700, 1750, 1800, 1850, 1900, 1950 or 2000 nm).

### Nucleic Acid Portion of Probe

The metal nanoclusters are associated with a nucleic acid to form the probe. The nucleic acid portion of the probe can be 3, 4, 5, 6, 7, 8, 9, 10, 11, 12, 13, 14, 15, 16, 17, 18, 19, 20, 21, 22, 23, 24, 25, 26, 27, 28, 29, 30, 31,32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139 140, 141, 142, 143, 144, 145, 146, 147, 148, 149, or 150 nucleotides long, or longer. In a particular embodiment, the nucleic acid is between 3-150 nucleotides long.

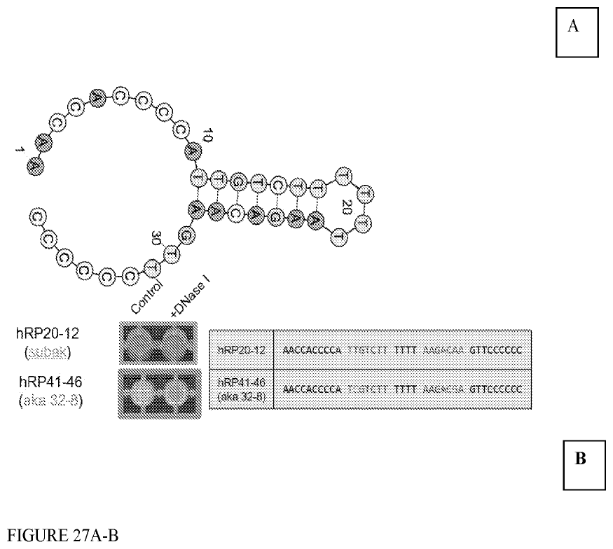

The nucleic acid portion of the probe can comprise secondary structure. An example of a hairpin can be seen in FIG. 27A-B. It is noted that any probe which is capable of working with the metal nanoclusters using the methods described herein, regardless of the shape. Examples of secondary structure include, but are not limited to, double helices, stem-loop structures, hairpins, psuedoknots, and G-quadruplexes. It has been found that hairpin structures are particularly useful, and therefore the Subak probes, and modified Subak probes, described herein can be used in a hairpin form, as shown in FIG. 27A.

The nucleic acid portion of the probe can be DNA or RNA, and can comprise synthetic components, such as analogs. A nucleotide analog is a nucleotide which contains some type of modification to either the base, sugar, or phosphate moieties. Modifications to nucleotides are well known in the art and would include for example, 5 methylcytosine (5 me C), 5 hydroxymethyl cytosine, xanthine, hypoxanthine, and 2 aminoadenine as well as modifications at the sugar or phosphate moieties. There are many varieties of these types of molecules available in the art and available herein.

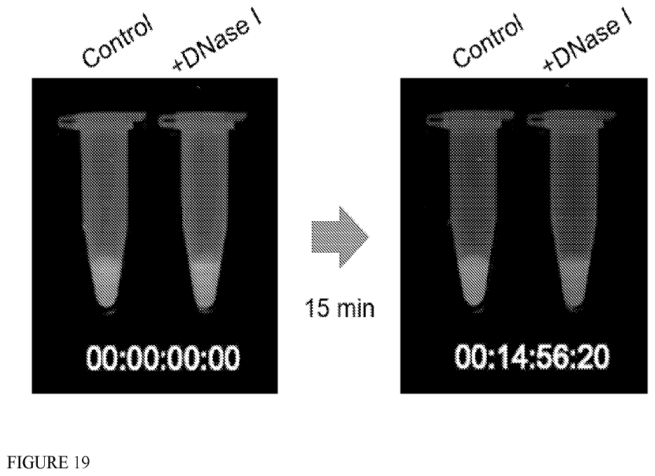

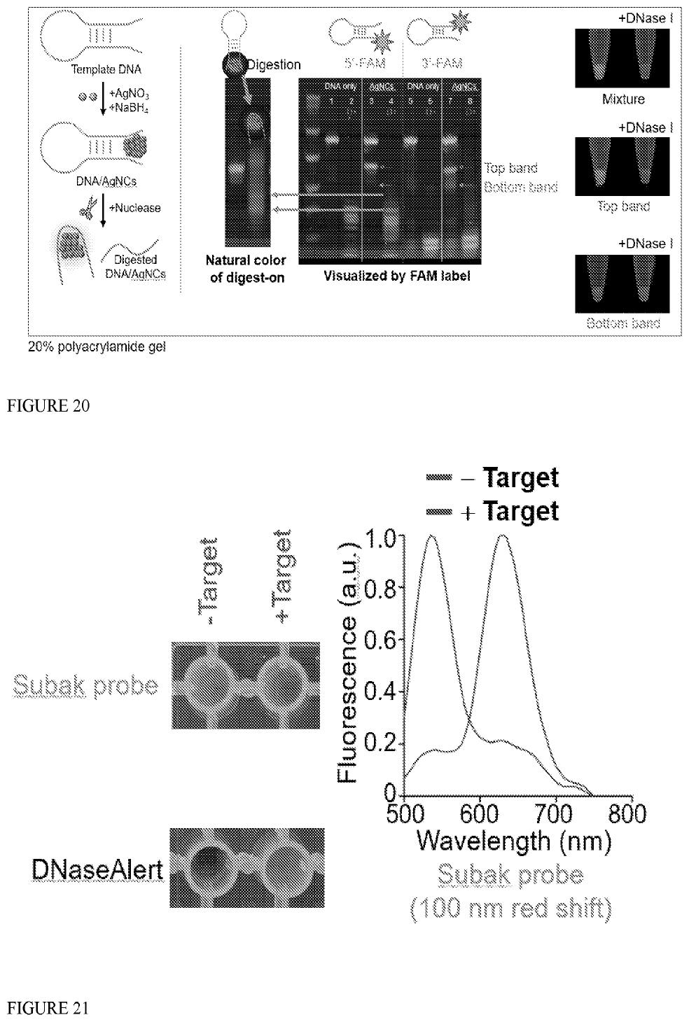

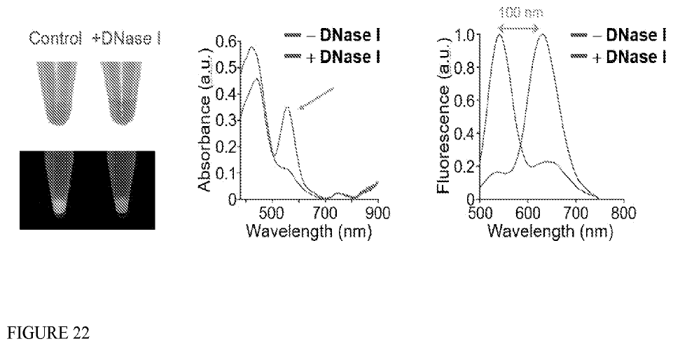

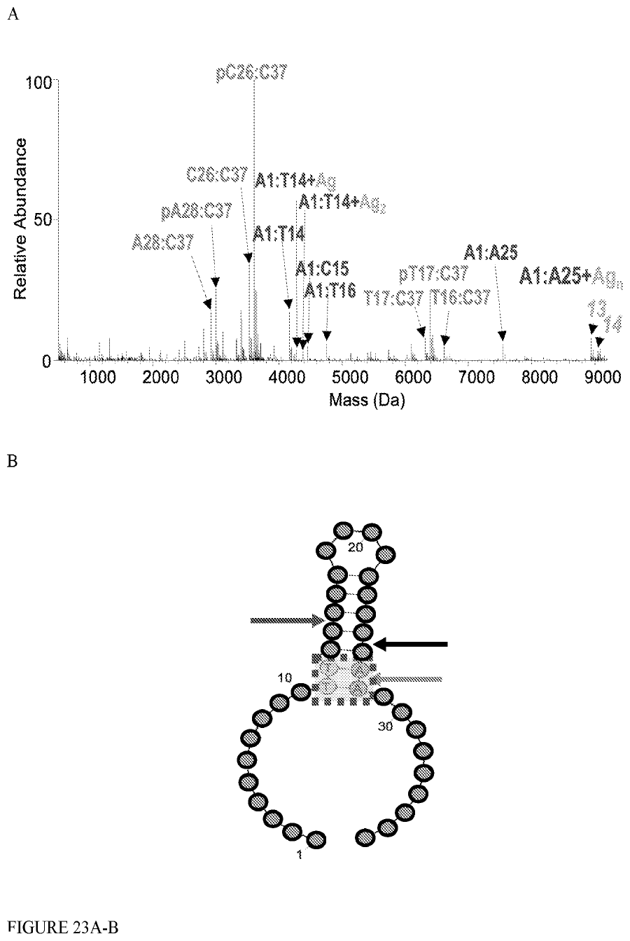

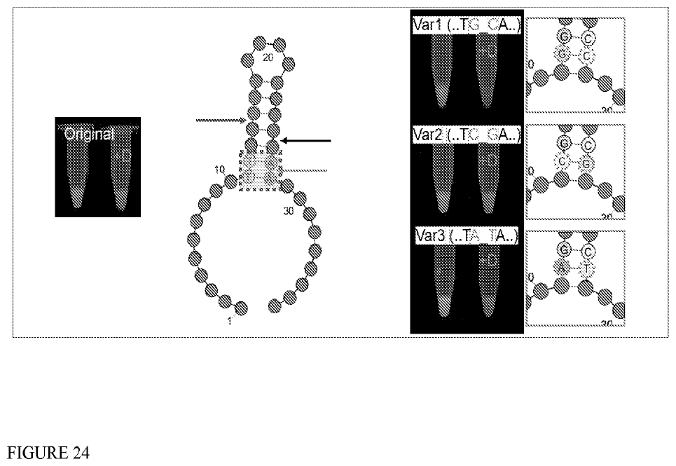

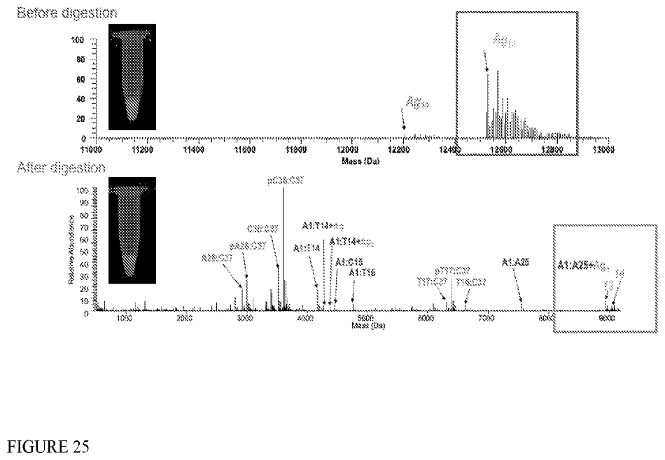

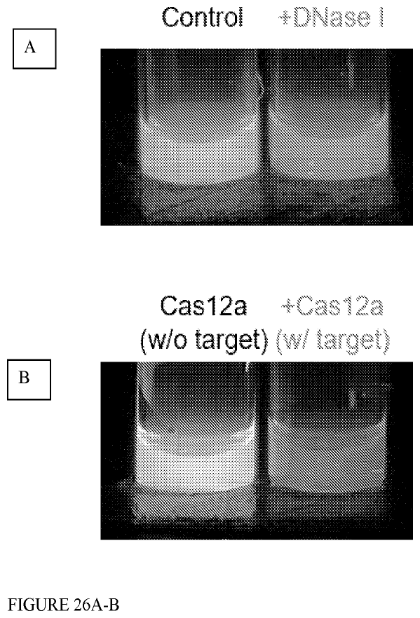

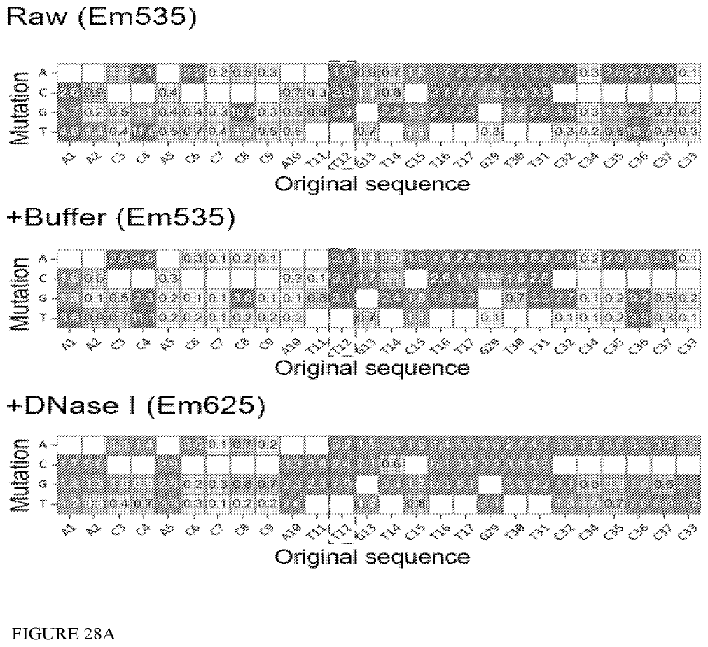

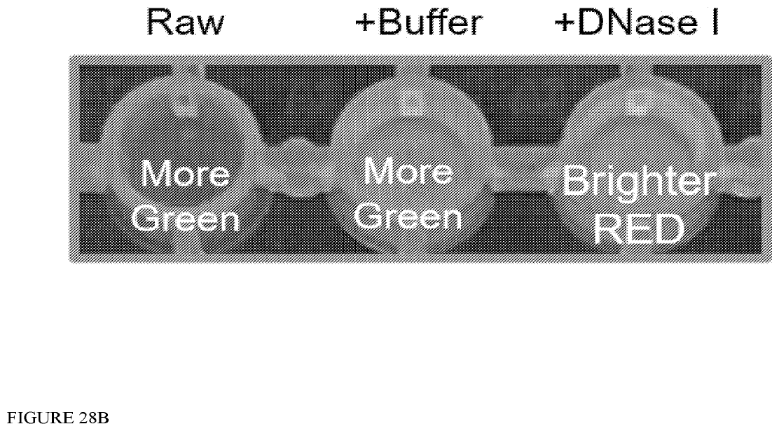

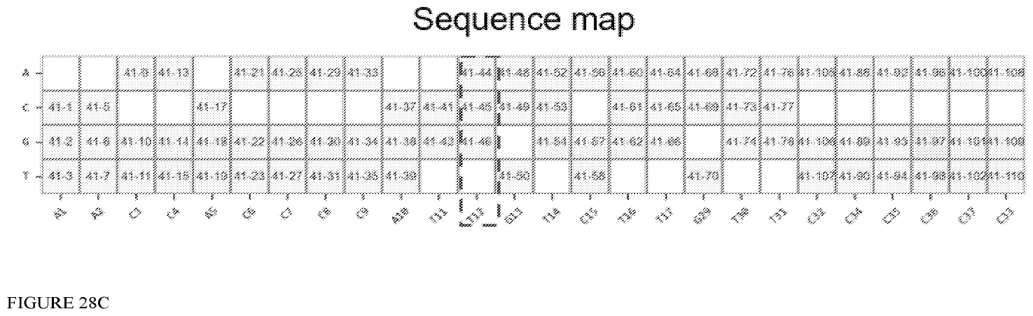

One specific example of a probe which can be used is a “Subak probe.” These probes are described in detail in FIGS. 19-22, for example. Subak probes can comprise SEQ ID NO: 9 or 10, for example, or a nucleic acid with one, two, three, four, five, six, seven, eight, nine, or ten or more changes in sequence as compared to SEQ ID NO: 9. For example, disclosed herein is a sequence with 80, 81, 82, 83, 84, 85, 86, 87, 88, 89, 90, 91, 92, 93, 94, 95, 96, 97, 98, or 99% identity to SEQ ID NO: 9. Examples include SEQ ID NOS: 10-111. Specifically contemplated are hRP41-46 (SEQ ID NO: 10), hRP41-54 (T14G) (SEQ ID NO: 63) and hRP41-105 (C32A) (SEQ ID NO: 106), which specifically show highly detectable levels of fluorsecence. The relative fluorescent intensities are shown in FIG. 28, along with the position of variation in each variant.

### Uses of Nucleic Acid-Metal Nanocluster Probes

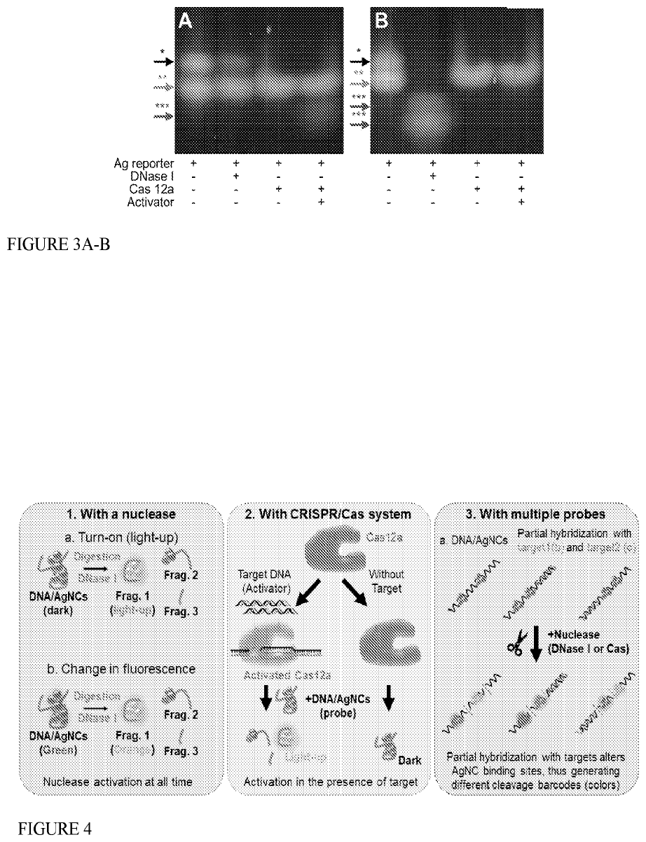

The probes disclosed herein can be used in a variety of contexts for a variety of reasons to aid in nucleic acid detection. Importantly, all of these uses revolve around the “digestion-and-light-up” concept outlined herein. Three of the major uses include, but are not limited to, 1) with a CRISPR/Cas detection system, such as DETECTR and SHERLOCK, 2) with a nuclease, and with 3) hybridization of probes directly to target nucleic acids, followed by a cleavage event. These three uses are illustrated in FIG. 4.

### Nucleases

Contemplated herein is the use of the nucleic acid-metal nanocluster probes disclosed herein to detect the presence of a nuclease, or to detect a cleavage event in a sample. This is illustrated in FIG. 2A-F. One of skill in the art will appreciate that the probe can be designed with the specific nuclease or cleavage event to be detected in mind. The probes disclosed herein can be used with any type of nuclease, either exonucleases or endonucleases, and either specific or non-specific nucleases. They can also be used with DNase or RNase. Therefore, the nucleic acid portion of the probe can comprise a specific recognition site when used with a nuclease that requires the same, or can have a general length or secondary structure which will aid in its cleavage by a specific nuclease. Furthermore, the nucleic acid can be designed to be resistant to some nucleases, such as by the use of various modifications or secondary structure, as discussed below. This can be used to exclude digestion/restriction by certain nucleases, while permitting restriction/digestion by other nucleases. It is noted that the nuclease can be a Cas molecule which is specific for a given sequence, such as Cas9.

### CRISPR/Cas Detection Systems

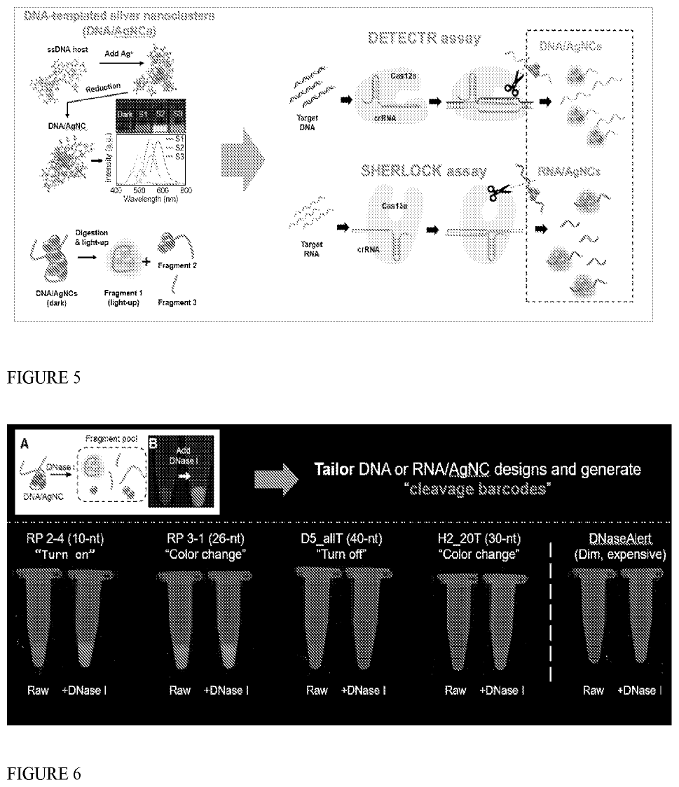

The nucleic acid-metal nanocluster probes disclosed herein can also be used in conjunction with a CRISPR/Cas based detection system such as DETECTR or SHERLOCK. When this is the case, the probe need not be complementary to the target nucleic acid, as the Cas molecule will cleave indiscriminately when the presence of the target nucleic acid is detected. This is illustrated in FIGS. 1A-C and FIG. 5, for example. One of skill in the art will understand how to design probes such that they are useful with a CRISPR/Cas based detection system. In some cases, it is not desirable for the nucleic acid portion of the probe to have significant complementarity to the target, as it may interfere with the cleavage/digestion of the probe. This is because the CRISPR/Cas system separately detects the target nucleic acid, then indiscriminately cleaves the probe. In this case, the probe can be designed so that it does not hybridize with the target or other off-target nucleic acids which may be present. These probes can also be used in a multiplex assay, so that more than one probe, wherein each probe is associated with a different nucleic acid portion and/or a different metal nanocluster arrangement, is used.

### Differential Fluorescence Detection

The probes containing a fluorescent metal nanocluster disclosed herein can also be used to detect different target nucleic acids in the same reaction. When the probes hybridize to the target, binding of each target nucleic acid to the nucleic acid-metal nanocluster probe can cause shifts in the metal nanocluster localization pattern. After the target binds, the nanocluster is relocated. Following this event, digestion of the nucleic acid can take place. Because the silver nanoclusters are located at different places on the probes due to differential binding of different targets, digestion patterns are not the same. This is shown as a “spectral pattern,” or “cleavage barcode”. So the “cleavage barcode” is cleavage that generates different spectrum emission patterns as a result of different target-probe interactions.

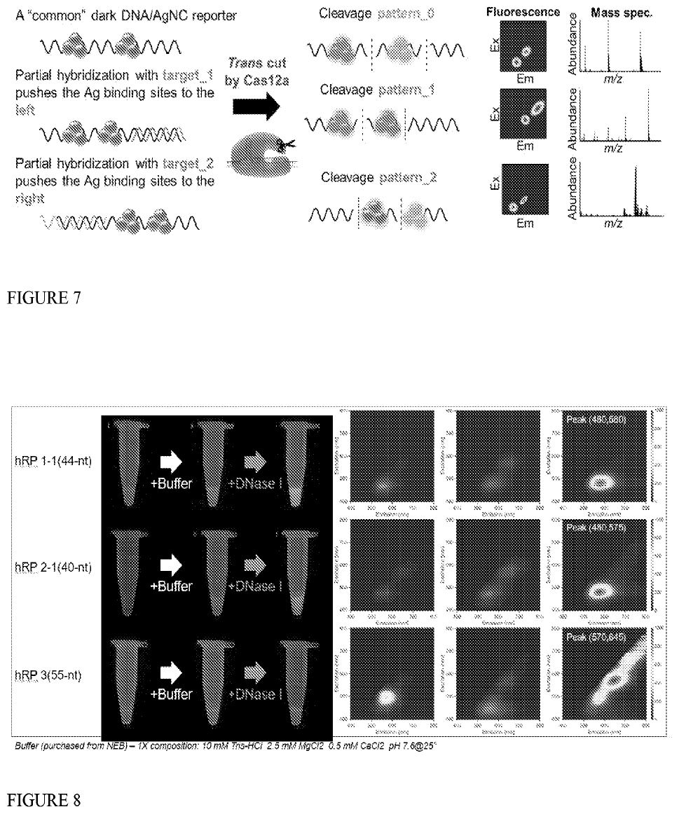

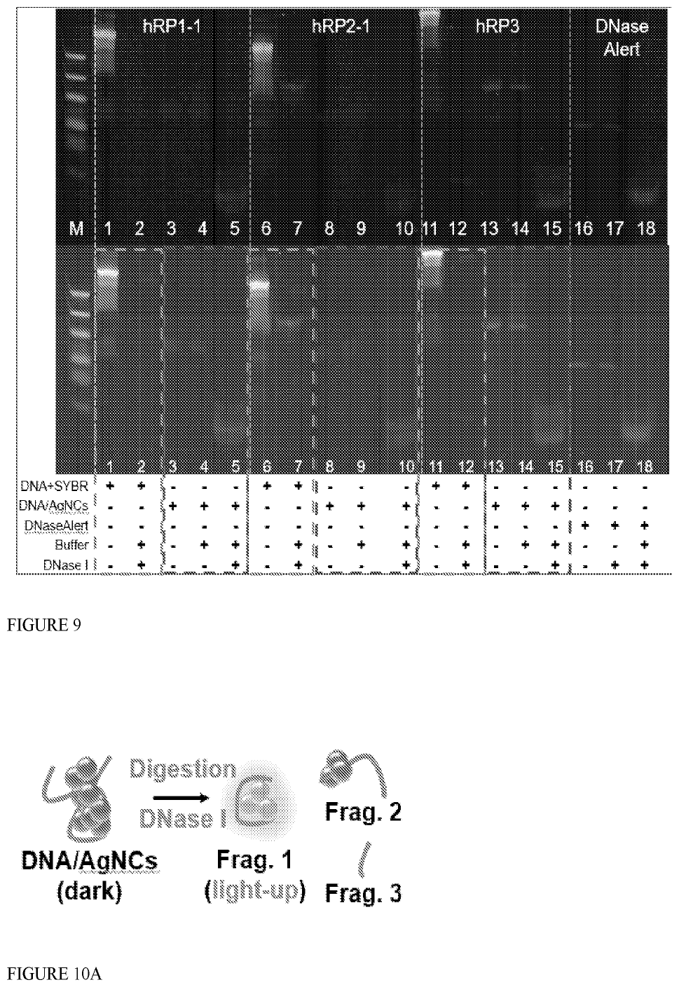

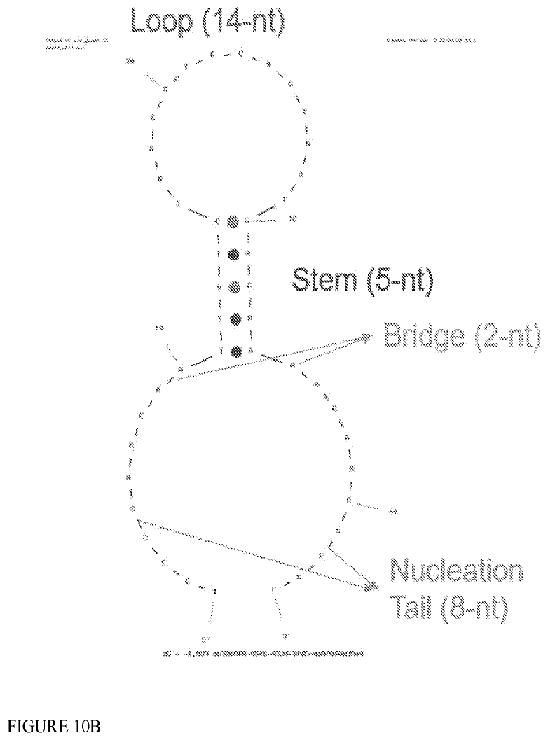

This system can be used with a nuclease, such as DNase I, or with the CRISPR/Cas detection methods described above. It is noted that 1, 2, 3, 4, 5, 6, 7, 8, 9, 10, or more probes can be used in the same reaction. Also, each probe can be specific for 1, 2, 3, 4, 5, or more different targets. Specifically, a target can partially bind the fluorescent metal nanocluster reporter and alter the nanocluster binding site on the reporter. This makes the probe show a different spectrum pattern when it is trans cut differently, such as by activated CRISPR/Cas. This trans cleavage pattern can be altered by savvy selection of the target and the reporter sequences as illustrated in FIG. 4. This presence can result in differential fluorescence, meaning that a molecule that was fluorescing at one wavelength may begin to fluoresce at a different wavelength. This change in fluorescence pattern can be detected and used to determine binding of a target, or binding of one target versus another. These examples are illustrated in FIGS. 6 and 7, for example.

The probes disclosed herein are capable of wide ranges of color shifts upon cleavage events. They can be “dark” to start with, and then, upon cleavage, can undergo a shift so that a detectable color can be viewed. Alternatively, the probe can shift from one detectable color to another. Examples of detectable color shifts can be that a molecule which absorbs red, pink, orange, yellow, green, blue, or violet, or any other color, or any color in between these colors, can shift so that they have a different visible emission color upon cleavage (that cleavage is related to probe-target interactions, which is called “partial hybridization” in FIG. 5). As stated above, this color shift can be sensitive enough to detect the binding of one target versus another, or the binding of more than one target versus only one target.

Since the nucleic acid portion of the probe and the target interact through hybridization, this is a tunable aspect of the invention. The term hybridization is defined above, and typically means a sequence driven interaction between at least two nucleic acid molecules. Parameters for selective hybridization between two nucleic acid molecules are well known to those of skill in the art. For example, in some embodiments selective hybridization conditions can be defined as stringent hybridization conditions. Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their Kd, or where only one of the nucleic acid molecules is 10 fold or 100 fold or 1000 fold or where one or both nucleic acid molecules are above their Kd.

It is understood that there are a variety of methods herein disclosed for determining the level of hybridization between two nucleic acid molecules. Because the metal nanoclusters disclosed herein are highly tunable, they can detect the strength of a hybridization reaction between the target and the nucleic acid portion of the probe. Detection of hybridization is discussed in more detail in the “methods” section below.

### Compositions and Kits

The nucleic acid-metal nanocluster probes disclosed herein can be used within a composition, so that components other than the nucleic acid-metal nanocluster probe can be present. In other words, the compositions can comprise other components which are needed or desired for nucleic acid detection. These components can also be used in kit form. When provided in a kit, other components which aid in the methods disclosed herein, or which provide convenience to the user, can also be included. One of skill in the art will recognize which components can be included in such a kit.

### CRISPR Cas Systems

In addition to the nucleic acid-metal nanocluster probes, the composition can also comprise a CRISPR/Cas system for nucleic acid detection and cleavage of the probe. In general, a CRISPR-Cas or CRISPR system refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system).

In certain embodiments, a protospacer adjacent motif (PAM) or PAM-like motif directs binding of the effector protein complex as disclosed herein to the target locus of interest. In some embodiments, the PAM may be a 5′ PAM (i.e., located upstream of the 5′ end of the protospacer). In other embodiments, the PAM may be a 3′ PAM (i.e., located downstream of the 5′ end of the protospacer). The term “PAM” may be used interchangeably with the term “PFS” or “protospacer flanking site” or “protospacer flanking sequence”.

The CRISPR effector protein may recognize a 3′ PAM. In certain embodiments, the CRISPR effector protein may recognize a 3′ PAM which is 5′H, wherein H is A, C or U. In certain embodiments, the effector protein may be Leptotrichia shahii C2c2p, more preferably Leptotrichia shahii DSM 19757 C2c2, and the 3′ PAM is a 5′ H.

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to a RNA polynucleotide being or comprising the target sequence. In other words, the target RNA may be a RNA polynucleotide or a part of a RNA polynucleotide to which a part of the gRNA, i.e. the guide sequence, is designed to have complementarity and to which the effector function mediated by the complex comprising CRISPR effector protein and a gRNA is to be directed. In some embodiments, a target sequence is located in the nucleus or cytoplasm of a cell.

RNA-targeting effector proteins include CRISPR/Cas Class II enzymes. Examples include Type V and Type VI enzymes, such as those used with the SHERLOCK and DETECTR methods of nucleic acid detection. These include, but are not limited to, Cas14, Cas13a, Cas13b, and Cas12a, among others. In certain embodiments, more than one CRISPR system effector protein is provided in the same composition.

Also disclosed herein for use with the compositions is a Cas transgenic cell, in which one or more nucleic acids encoding one or more guide RNAs are provided or introduced operably connected in the cell with a regulatory element comprising a promoter of one or more gene of interest. As used herein, the term “Cas transgenic cell” refers to a cell, such as a eukaryotic cell, in which a Cas gene has been genomically integrated. The nature, type, or origin of the cell are not particularly limiting according to the present invention. Also the way the Cas transgene is introduced in the cell may vary and can be any method as is known in the art. In certain embodiments, the Cas transgenic cell is obtained by introducing the Cas transgene in an isolated cell. In certain other embodiments, the Cas transgenic cell is obtained by isolating cells from a Cas transgenic organism.

In certain aspects the invention involves vectors, e.g. for delivering or introducing in a cell Cas and/or RNA capable of guiding Cas to a target locus (i.e. guide RNA), but also for propagating these components (e.g. in prokaryotic cells). A used herein, a “vector” is a tool that allows or facilitates the transfer of an entity from one environment to another. It is a replicon, such as a plasmid, phage, or cosmid, into which another DNA segment may be inserted so as to bring about the replication of the inserted segment. Generally, a vector is capable of replication when associated with the proper control elements. In general, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell).

### Buffers

The nucleic acid-metal nanocluster probe disclosed herein can further comprise a buffer. This buffer can stabilize the fluorescent metal nanocluster and associated nucleic acid sequence. For example, the buffer can comprise about 5, 6, 7, 8, 9, 10, 11, 12, 13, 14, 15, 16, 17, 18, 19, 20, 21, 22, 23, 24, or 25 mM of sodium phosphate. The pH of the buffer with sodium phosphate can be about 6.0, 6.1, 6.2, 6.3, 6.4, 6.5, 6.6, 6.7, 6.8, 6.9, 7.0, 7.1, 7.2, 7.3, or 7.5. Preferably, the buffer comprising sodium phosphate is present in the composition at about 20 mM at a pH of about 6.6.

The buffer can further comprise Tris-HCl. This can be present at 5, 6, 7, 8, 9, 10, 11, 12, 13, 14, or 15 mM in the buffer. The buffer with HCl can be at a pH of about 7.0, 7.1, 7.2, 7.3, 7.4, 7.5, 7.6, 7.7, 7.8, 7.9, 8.0, 8.1, or 8.2. Preferably, the buffer comprising HCl is present at about 10 mM Tris-HCl at a pH of about 7.6.

The buffer can further comprise ammonium acetate. The ammonium acetate can be present at 5, 6, 7, 8, 9, 10, 11, 12, 13, 14, 15, 16, 17, 18, 19 or 20 mM. The pH of the buffer with ammonium acetate can be 6.5, 6.6, 6.6, 6.7, 6.8, 6.9, 7.0, 7.1, 7.2, 7.3, or 7.5. Preferably, the buffer comprising ammonium acetate is present at about 10 mM at a pH of about 7.0. This buffer can further comprise MgCl₂ at about 2.0, 2.1, 2.2, 2.3, 2.4, 2.5, 2.6, 2.7, 2.8, 2.9, or 3.0 mM, and can further comprise CaCl₂ at about 0.1, 0.2, 0.3, 0.4, 0.5, 0.6, 0.7, 0.8, 0.9, or 1.0 mM CaCl₂. Preferably, the buffer comprises MgCl₂ at about 2.5 mM and CaCl₂ at about 0.5 mM. One of skill in the art will appreciate that each nuclease will require slightly different ingredients in its buffer in order to maximize its enzyme activity.

### Methods

As discussed above, there are envisioned at least three ways that the nucleic acid-metal nanoclusters disclosed herein can be used to detect the presence of a nucleic acid (or the presence of a nuclease, in some cases). These are illustrated in FIG. 4.

In the first method disclosed herein, a CRISPR/Cas system is used to detect the presence of a nucleic acid is discussed above. In this method for detecting a target nucleic acid in a sample, the method comprising (a) contacting the sample with at least one ribonucleoprotein (RNP) comprising a guide molecule specific for at least one target nucleic acid and an effector molecule which is capable of cleaving a probe upon binding of the guide molecule in the presence of target nucleic acids; and at least one probe comprising a nucleic acid sequence and a fluorescent metal nanocluster associated with the nucleic acid sequence; and (b) measuring a detectable signal produced by cleavage of the probe by the effector molecule upon binding of the guide molecule, thereby detecting the target nucleic acid. the guide system comprises a guide nucleic acid.

In this method, the probe produces a first detectable signal prior to being cleaved and a second detectable signal after cleavage of the probe. As discussed above, the probe may produce no detectable signal prior to being cleaved and a detectable signal after cleavage of the probe. Alternatively, the probe may produce a detectable signal prior to being cleaved and no detectable signal after cleavage of the probe. The target nucleic acid can hybridize to the probe. In some instances, more than one target can hybridize to the same probe. The method can be done in multiplex, so that more than one probe is used. Each probe can be specific for a different target nucleic acid. The target nucleic acid can be amplified before or during the step of measuring a detectable signal.

The sample can comprise DNA molecules from a cell lysate. The sample can also comprise whole cells. These cells can be eukaryotes or prokaryotes. The method can comprise measuring the detectable signal to generate a test measurement; measuring a detectable signal produced by a reference sample or cell to generate a reference measurement; and then comparing the test measurement to the reference measurement to determine an amount of target nucleic acid present in the sample.

In the second method envisioned for use with the nucleic acid-metal nanocluster detection probes discussed herein, the method comprises detecting nuclease activities, wherein the method comprises (a) contacting the sample with: a nuclease which is capable of cleaving a probe upon binding of the guide molecule; and at least one probe comprising a nucleic acid sequence and a fluorescent metal nanocluster associated with the nucleic acid sequence; and (b) measuring a detectable signal produced by cleavage of the probe by the nuclease, thereby detecting the target nucleic acid.

Various nucleases that can be used with this method are discussed above, and can include endonucleases, exonucleases, and DNase I or RNase I, among others. The nuclease can also be a Cas nuclease which is specific for a given sequence, or can be engineered to be specific for a specific sequence, such as Cas9.

As discussed above, the probe can produce a first detectable signal prior to being cleaved and a second detectable signal after cleavage of the probe. Alternatively, the probe can produce no detectable signal prior to being cleaved and a detectable signal after cleavage of the probe. In yet another embodiment, the probe can produce a detectable signal prior to being cleaved and no detectable signal after cleavage of the probe. The method can be done in multiplex, so that more than one probe is used. Each probe can be specific for a different target nucleic acid. The target nucleic acid can be amplified before or during the step of measuring a detectable signal.

The third method envisioned in which the nucleic acid-metal nanocluster probes can be used is by detection by hybridization to the nucleic acid portion of the probes. Therefore, disclosed is a method of detecting the presence of multiple target nucleic acids in a single reaction, the method comprising: (a) contacting the sample with a probe, wherein said probe comprises: at least two nucleic acid sequences which are capable of hybridizing with two or more target nucleic acids; at least one fluorescent metal nanocluster associated with the nucleic acid sequence; and (b) measuring a detectable signal, wherein hybridization of one probe gives a first signal, hybridization of a second probe gives a second signal, and hybridization of both probes gives a third signal; thereby detecting the presence of multiple target nucleic acids. In some embodiments, more than one probe can be present, such as 2, 3, 4, 5, 6, 7, 8, 9, 10, or more probes. These probes can hybridize with different target nucleic acids. The different probes can be associated with different fluorescent molecules. This is discussed in more detail above.

### EXAMPLES

### Example 1: A DNA-templated Silver Nanocluster Probe That Changes Fluorescence Upon Digestion

### Materials

Oligonucleotides with standard desalting and crRNA were purchased from Integrated DNA Technologies, Inc. (USA), IDT, and suspended by DNase-free water from IDT. 99.9999% Silver nitrate (Cat No. 204390) and 99.99% sodium borohydride (Cat No. 480886) were purchased from Sigma Aldrich (USA). DNaseAlert (Cat No. Nov. 4, 2002-04) was purchased from IDT, DNase I (Cat No. M0303L) and EnGen Lba Cas12a (Cpf1) (Cat No. M0653T) were purchased from New England BioLabs (USA). Ammonium acetate (0219875980, MP Biomedicals (USA)) was diluted before use, in which the final solutions were 20 mM and pH 7. SureCast ammonium persulfate (Cat No. HC2005), SureCast acrylamide solution (Cat No. HC2040), and SureCast TEMED (Cat No. HC2006) were ordered from ThermoFisher Scientific (USA).

### Methods

### DNA-templated Silver Nanocluster Preparation

The oligonucleotides were suspended at 500 μM in DNase-free water and then thawed in water at 70° C. for 2 minutes and cooled for at least 5 minutes. To make 1 mL of solution, 80 μL of 500 μM DNA was mixed with 40 μL of 5 mM silver nitrate (AgNO₃) solution, 500 μL of 20 mM ammonium acetate solution (NH₄OAc) and 370 μL of deionized water, and then the mixture was vortexed and centrifuged for 1 minute at 14,000 RCF. After equilibration for 20 minutes, the mixture was reduced with 10 μL of 10 mM freshly prepared sodium borohydride (NaBH₄) to form DNA-templated silver nanoclusters. The solution was then vortexed and centrifuged again for 1 minute at 14,000 RCF. The final concentrations are 40 μM DNA, 10 mM NH₄OAc, 200 μM AgNO₃, and 100 μM NaBH₄. The product was stored at 4° C.

### Fluorescence Measurement

All fluorescence emission and excitation spectra were acquired using a FluoroMax-4 spectrofluorometer from Horiba Scientific. Each 120 μL sample was placed in a 100 μL quartz cuvette (16.100F-Q-10/Z15, Starna Cells) for fluorometer measurements. For 2D measurements, both emission and excitation were scanned from 400 to 800 nm using 5 nm in slit size, 5 nm in increment step, and 0.1 s in integration time. Final results were visualized by a customized Python code.

### DNase I Digestion

The 10× buffer for DNase I experiment was prepared with 100 mM ammonium acetate mixed with 100 mM magnesium acetate. It was filtered and autoclaved before use, and its pH was adjusted to 7.0 before the experiment. 5 μL of DNase I (1 U/μL) was first mixed with 5 μL of 10× buffer with 15 μL of deionized water (18.2 MΩ-cm). Then, 25 μL of 40 μM DNA/AgNCs were loaded and the sample tube was incubated at 37° C. for 30 minutes. For DNaseAlert validation, 25 μL of 4 μM reporter was loaded instead of DNA/AgNCs.

### CRISPR Cas12a Digestion

The same 10× buffer was used for CRISPR/Cas12a. 10 μL of 2 μM Cas12a was mixed with 5 μL of 10× buffer and 4 μL deionized water (18.2 MS2-cm). Then, 5 μL of 5 μM gRNA was loaded and the solution was pre-incubated at room temperature for 10 minutes. 1 μL of 800 nM substrate DNA to activate Cas12a enzyme was loaded and incubated at 37° C. for 20 minutes. 25 μL of 40 μM DNA/AgNCs were loaded and the sample tube was incubated at 37° C. for 30 minutes. For DNaseAlert validation, 25 μL of 4 μM reporter was loaded instead of DNA/AgNCs.

Polyacrylamide gel electrophoresis (PAGE)

20-25% homemade polyacrylamide gels were prepared. 20 μL of sample was loaded into each well. The sample includes 10× loading TBE buffer and DNA/AgNCs were concentrated up to 250 μM before running to maximize the visualization. Then, the gel was ran at 75V for 400 minutes and visualized under UV excitation using a transilluminator.

### Results

Four different DNA/AgNCs with DNase I as compared to DNaseAlert (FIG. 2) were examined. They all showed the significant fluorescence changes with DNase I but in different ways. Reporter 1, GAT CCC CAA C (SEQ ID NO: 8), showed turn-on-based changes as expected, since the fragment of dark DNA/AgNCs by DNase I seemed to become bright. Different types of fluorescence changes including turn-off (Reporter 2, GGC CCT GCG ATT TTT TTT TTT TTT TTT TTT TTT TTT TTT T (SEQ ID NO: 1), FIG. 2C), and color change from orange to yellow (Reporter 3, GGG TTA GGG TCC CCC CAC CCT TAC CC (SEQ ID NO: 2), FIG. 2D) and from red to yellowish green (Reporter 4, AAT CCC CCC ATT TTT TTT TTT TTT TTT TTT, (SEQ ID NO: 3) FIG. 2E) were observed. All these changes are more clear and evident than that from DNaseAlert.

While metal cation preference of Cas enzymes in trans cleavage has been tested for Ca, Co, Cu, Mg, Mn, Ni, and Zn₉, DNA interacting Ag ions or Ag atoms have not been tested. It was hypothesized that by controlling Ag and other DNA-interacting metals in the reaction, the trans cleavage pattern can be manipulated on a given DNA/AgNC reporter probe, even generating a “cleavage barcode” that corresponds to the DNA target sequence selection. For the traditional FRET reporter probe, when it is cleaved, the donor dye's fluorescence is recovered, no matter how the probe is cut. However, the novel DNA/AgNC probes are different. As shown in FIG. 3, polyacrylamide gel experiments were run to validate the digestion on the two DNA/AgNC probes. Among tested candidates. Reporter 5 (10-nt DNA/AgNCs, AAT CCC CCC A, SEQ ID NO: 4) and Reporter 6 (26-nt DNA/AgNCs, GGG TTA GGG TCC CCC CAC CCT TAC CC, SEQ ID NO: 2) were labeled with 6-FAM dyes at the 5′ end for visualization. They both showed two bands (*, ** in FIG. 3) without nucleases, but the top band (*) disappeared or got dimmer with DNase I or CRISPR-Cas12a. Interestingly, we could see the different digestion impact on the probes varying by their template sequence length. FIG. 3A is in-gel validation with a short

Reporter 5, in which the collateral cleavage activity by the activated Cas12a was clearly validated (***), but DNase I only made the top band (*) dimmer. As expected, non-activated Cas12a could not generate the third band (***). FIG. 3B showed the different cleavage patterns with a long Reporter 6, in which DNase I clearly cut the reporter probes generating the third and fourth bands (***), but Cas12a did not generate any additional bands regardless of the existence of activator strands.

### Conclusions

It was shown that the dual-labeled FRET probes in CRISPR/Cas assays could be replaced to generate cleavage barcodes for multiplexing. They are not only more sensitive but also cost-effective compared to the conventionally available FRET probes, DNaseAlert. Using gel electrophoresis and mass spectrometry, one could observe “orthogonal” cleavage patterns reflecting different target DNAs with only one universal DNA/AgNC probe and CRISPR/Cas12a enzyme, thus enabling multiplexing. A simple amplification-free nucleic acid detection for SARS-CoV-2 virus was shown, and it was envisioned that this technique can be developed for multiplex nucleic acid biosensors.

### Example 2: Subak Probes

A Subak probe with a hairpin DNA template can be used with the methods and systems described herein. One example of a Subak probe is a 37-nt ssDNA (AACCACCCCATTGTCTTTTTTAAGACAAGTTCCCCCC, SEQ ID NO: 9). More than 100 variants were tested based on the original sequence by changing one or two bases to another at different locations (i.e. A2C, A2T, T12C). As more sequences that have similarity with the original template were found to have the same activation mechanism, it has been found that a template in the form of a hairpin DNA can be used as Subak probes. An example of a modified Subak probe comprises AACCACCCCATCGTCTTTTTTAAGACGAGTTCCCCCC (T12C and A27G, SEQ ID NO: 10). Furthermore, it has been shown that some bases at specific locations (A1,C3,C4, C7, C8, C9, G13, C15, C33, C34, C35, C36) cannot be replaced because they are either related to either the nucleation site of silver nanoclusters or the cleavage site by a nuclease. However, as can be seen in FIG. 28A-C, various other substitutions can be made and highly functional probes can be obtained.

### REFERENCES

(1) Sheridan, C., Fast, portable tests come online to curb coronavirus pandemic. Nature Biotechnology 2020, 10.
(2) Carter, L. J.; Garner, L. V.; Smoot, J. W.; Li, Y.; Zhou, Q.; Saveson, C. J.; Sasso, J. M.; Gregg, A. C.; Soares, D. J.; Beskid, T. R., Assay Techniques and Test Development for COVID-19 Diagnosis. ACS Publications: 2020.
(3) Cervia, C.; Nilsson, J.; Zurbuchen, Y.; Valaperti, A.; Schreiner, J.; Wolfensberger, A.; Raeber, M. E.; Adamo, S.; Emmenegger, M.; Hasler, S., Systemic and mucosal antibody secretion specific to SARS-CoV-2 during mild versus severe COVID-19. bioRxiv 2020.
(4) Cheng, M. P.; Papenburg, J.; Desjardins, M.; Kanjilal, S.; Quach, C.; Libman, M.; Dittrich, S.; Yansouni, C. P., Diagnostic testing for severe acute respiratory syndrome-related coronavirus-2: A narrative review. Annals of Internal Medicine 2020.
(5) Nie, S.; Roth, R. B.; Stiles, J.; Mikhlina, A.; Lu, X.; Tang, Y.-W.; Babady, N. E., Evaluation of Alere i Influenza A&B for rapid detection of influenza viruses A and B. Journal of Clinical Microbiology 2014, 52 (9), 3339-3344.
(6) Chen, J. S.; Ma, E.; Harrington, L. B.; Da Costa, M.; Tian, X.; Palefsky, J. M.; Doudna, J. A., CRISPR-Cas12a target binding unleashes indiscriminate single-stranded DNase activity. Science 2018, 360 (6387), 436-439.
(7) East-Seletsky, A.; O'Connell, M. R.; Knight, S. C.; Burstein, D.; Cate, J. H.; Tjian, R.; Doudna, J. A., Two distinct RNase activities of CRISPR-C2c2 enable guide-RNA processing and RNA detection. Nature 2016, 538 (7624), 270-273.
(8) Gootenberg, J. S.; Abudayyeh, O. O.; Lee, J. W.; Essletzbichler, P.; Dy, A. J.; Joung, J.; Verdine, V.; Donghia, N.; Daringer, N. M.; Freije, C. A.; Myhrvold, C.; Bhattacharyya, R. P.; Livny, J.; Regev, A.; Koonin, E. V.; Hung, D. T.; Sabeti, P. C.; Collins, J. J.; Zhang, F., Nucleic acid detection with CRISPR-Cas13a/C2c2. Science 2017, 356 (6336), 438-+.
(9) Gootenberg, J. S.; Abudayyeh, O. O.; Kellner, M. J.; Joung, J.; Collins, J. J.; Zhang, F., Multiplexed and portable nucleic acid detection platform with Cas13, Cas12a, and Csm6. Science 2018, 360 (6387), 439-444.
(10) Broughton, J. P.; Deng, X.; Yu, G.; Fasching, C. L.; Servellita, V.; Singh, J.; Miao, X.; Streithorst, J. A.; Granados, A.; Sotomayor-Gonzalez, A., CRISPR-Cas12-based detection of SARS-CoV-2. Nature Biotechnology 2020, 1-5.
(11) Joung, J.; Ladha, A.; Saito, M.; Segel, M.; Bruneau, R.; Meei-li, W. H.; Kim, N.-G.; Yu, X.; Li, J.; Walker, B. D.; Greninger, A. L.; Jerome, K. R.; Gootenberg, J. S.; Abudayyeh, O. O.; Zhang, F., Point-of-care testing for COVID-19 using SHERLOCK diagnostics. medRxiv 2020.
(12) Kaminski, M. M.; Alcantar, M. A.; Lape, I. T.; Greensmith, R.; Huske, A. C.; Valeri, J. A.; Marty, F. M.; Klämbt, V.; Azzi, J.; Akalin, E., A CRISPR-based assay for the detection of opportunistic infections post-transplantation and for the monitoring of transplant rejection. Nature Biomedical Engineering 2020, 1-9.
(13) Ackerman, C. M.; Myhrvold, C.; Thakku, S. G.; Freije, C. A.; Metsky, H. C.; Yang, D. K.; Ye, S. H.; Boehm, C. K.; Kosoko-Thoroddsen, T.-S. F.; Kehe, J., Massively multiplexed nucleic acid detection using Cas13. 2020.
(14) Petty, J. T.; Ganguly, M.; Yunus, A. I.; He, C.; Goodwin, P. M.; Lu, Y.-H.; Dickson, R. M., A DNA-encapsulated silver cluster and the roles of its nucleobase ligands. Journal of Physical Chemistry C 2018.
(15) Obliosca, J. M.; Liu, C.; Yeh, H.-C., Fluorescent silver nanoclusters as DNA probes. Nanoscale 2013, 5, 8443-8461.
(16) Neacşu, V. A.; Cerretani, C.; Liisberg, M. B.; Swasey, S. M.; Gwinn, E. G.; Copp, S. M.; Vosch, T., Unusually large fluorescence quantum yield for a near-infrared emitting DNA-stabilized silver nanocluster. Chemical Communications 2020.
(17) Choi, S.; Dickson, R. M.; Yu, J. H., Developing luminescent silver nanodots for biological applications. Chem Soc Rev 2012, 41 (5), 1867-1891.
(18) Yeh, H.-C.; Sharma, J.; Han, J. J.; Martinez, J. S.; Werner, J. H., A DNA-silver nanocluster probe that fluoresces upon hybridization. Nano Letters 2010, 10 (8), 3106-3110.
(19) Obliosca, J. M.; Babin, M. C.; Liu, C.; Liu, Y.-L.; Chen, Y.-A.; Batson, R. A.; Ganguly, M.; Petty, J. T.; Yeh, H.-C., A complementary palette of NanoCluster beacons. ACS Nano 2014, 8 (10), 10150-10160.
(20) Petty, J. T.; Giri, B.; Miller, I. C.; Nicholson, D. A.; Sergev, O. O.; Banks, T. M.; Story, S. P., Silver clusters as both chromophoric reporters and DNA ligands. Analytical Chemistry 2013, 85, 2183-2190.
(21) Petty, J. T.; Sergev, O. O.; Nicholson, D. A.; Goodwin, P. M.; Giri, B.; McMullan, D. R., A silver cluster-DNA equilibrium. Analytical Chemistry 2013, 85 (20), 9868-9876.
(22) Del Bonis-O'Donnell, J. T.; Thakrar, A.; Hirschberg, J. W.; Vong, D.; Queenan, B. N.; Fygenson, D. K.; Pennathur, S., DNA-stabilized silver nanoclusters as specific, ratiometric fluorescent dopamine sensors. ACS Chemical Neuroscience 2017, 9 (4), 849-857.
(23) Cerretani, C.; Vosch, T., Switchable dual-emissive DNA-stabilized silver nanoclusters. Acs Omega 2019, 4 (4), 7895-7902.
(24) Yeh, H.-C.; Sharma, J.; Shih Ie, M.; Vu, D. M.; Martinez, J. S.; Werner, J. H., A fluorescence light-up Ag nanocluster probe that discriminates single-nucleotide variants by emission color. Journal of the American Chemical Society 2012, 134 (28), 11550-8.
(25) Petty, J. T.; Story, S. P.; Hsiang, J.-C.; Dickson, R. M., DNA-templated molecular silver fluorophores. Journal of Physical Chemistry Letters 2013, 4 (7), 1148-1155.
(26) Vosch, T.; Antoku, Y.; Hsiang, J. C.; Richards, C. I.; Gonzalez, J. I.; Dickson, R. M., Strongly emissive individual DNA-encapsulated Ag nanoclusters as single-molecule fluorophores. P Natl Acad Sci USA 2007, 104 (31), 12616-12621.
(27) Sharma, J.; Rocha, R. C.; Phipps, M. L.; Yeh, H. C.; Balatsky, K. A.; Vu, D. M.; Shreve, A. P.; Werner, J. H.; Martinez, J. S., A DNA-templated fluorescent silver nanocluster with enhanced stability. Nanoscale 2012, 4 (14), 4107-10.
(28) Huard, D. J.; Demissie, A.; Kim, D.; Lewis, D.; Dickson, R. M.; Petty, J. T.;

Lieberman, R. L., Atomic structure of a fluorescent Ag8 cluster templated by a multi-stranded DNA scaffold. Journal of the American Chemical Society 2018, 141 (29), 11465-11470.
(29) Petty, J. T.; Sergev, O. O.; Ganguly, M.; Rankine, I. J.; Chevrier, D. M.; Zhang, P., A segregated, partially oxidized, and compact Ag10 cluster within an encapsulating DNA host. Journal of the American Chemical Society 2016, 138 (10), 3469-3477.
(30) Cerretani, C.; Kanazawa, H.; Vosch, T.; Kondo, J., Crystal structure of a NIR-emitting DNA-stabilized Ag16 nanocluster. Angewandte Chemie 2019, doi.org/10.1002/anie.201906766.
(31) Obliosca, J. M.; Liu, C.; Batson, R. A.; Babin, M. C.; Werner, J. H.; Yeh, H.-C., DNA/RNA detection using DNA-templated few-atom silver nanoclusters. Biosensors 2013, 3 (2), 185-200.
(32) Sharma, J.; Yeh, H.-C.; Yoo, H.; Werner, J. H.; Martinez, J. S., A complementary palette of fluorescent silver nanoclusters. Chemical Communications 2010, 46, 3280-3282.
(33) Chen, Y.-A.; Obliosca, J. M.; Liu, Y.-L.; Liu, C.; Gwozdz, M. L.; Yeh, H.-C., NanoCluster Beacons enable detection of a single N6-methyladenine. Journal of the American Chemical Society 2015, 137 (33), 10476-10479.
(34) Juul, S.; Obliosca, J. M.; Liu, C.; Liu, Y.-L.; Chen, Y.-A.; Imphean, D. M.; Knudsen, B. R.; Ho, Y.-P.; Leong, K. W.; Yeh, H.-C., NanoCluster Beacons as reporter probes in rolling circle enhanced enzyme activity detection. Nanoscale 2015, 7, 8332-8337.
(35) Yeh, H.-C.; Sharma, J.; Han, J. J.; Martinez, J. S.; Werner, J. H., A beacon of light-a new molecular probe for homogeneous detection of nucleic acid targets. IEEE Nanotechnology Magazine 2011, 5 (2), 28-33.
(36) Petty, J. T.; Fan, C.; Story, S. P.; Sengupta, B.; Sartin, M.; Hsiang, J.-C.; Perry, J. W.; Dickson, R. M., Optically enhanced, near-IR, silver cluster emission altered by single base changes in the DNA template. J Phys Chem B 2011, 115 (24), 7996-8003.
(37) Petty, J. T.; Story, S. P.; Juarez, S.; Votto, S. S.; Herbst, A. G.; Degtyareva, N. N.; Sengupta, B., Optical sensing by transforming chromophoric silver clusters in DNA nanoreactors. Anal Chem 2011, 84 (1), 356-364.
(38) Petty, J. T.; Nicholson, D. A.; Sergev, O. O.; Graham, S. K., A near-infrared silver cluster optically signals oligonucleotide hybridization and assembles two DNA hosts. Anal Chem 2014.
(39) Ganguly, M.; Bradsher, C.; Goodwin, P.; Petty, J. T., DNA-Directed Fluorescence Switching of Silver Clusters. The Journal of Physical Chemistry C 2015, 119 (49), 27829-27837.
(40) Petty, J. T.; Sergev, O. O.; Kantor, A. G.; Rankine, I. J.; Ganguly, M.; David, F. D.; Wheeler, S. K.; Wheeler, J. F., Ten-atom silver cluster signaling and tempering DNA hybridization. Anal Chem 2015, 87 (10), 5302-5309.

1. A composition comprising:
a. a probe which can indicate the presence of a target nucleic acid, the probe comprising a nucleic acid sequence and a fluorescent metal nanocluster associated with the nucleic acid sequence; and
b. a nuclease capable of cleaving the nucleic acid sequence of the probe.

2. The composition of claim 1, wherein the composition further comprises a buffer that stabilizes the fluorescent metal nanocluster and associated nucleic acid sequence.

3. The composition of claim 1, wherein the probe comprises a nuclease recognition site.

4. The composition of claim 1, wherein the nucleic acid sequence of the probe is 3-150 nucleotides in length.

5. (canceled)

6. The composition of claim 1, wherein the nucleic acid sequence of the probe can comprise a secondary structure.

7. The composition of claim 6, wherein said secondary structure comprises a stem-loop (hairpin) structure.

8. The composition of claim 1, wherein the fluorescent metal nanocluster is a silver nanocluster or a silver-containing nanocluster.

9. The composition of claim 1, wherein the nuclease is part of an effector/guide system.

10. The composition of claim 9, wherein said effector/guide system is a CRISPR/Cas System.

11. The composition of claim 10, wherein said Cas molecule comprises Class II Cas enzymes such as Cas12, Cas13, or Cas14.

12. The composition of claim 1, wherein the nuclease can be an exonuclease and an endonuclease.

13. The composition of claim 1, wherein the probe is not capable of substantially hybridizing to the target nucleic acid under hybridization conditions.

14. The composition of claim 1, wherein the probe is a Subak probe.

15. The composition of claim 14, wherein the probe is selected from the group comprising SEQ ID NO: 9-111.

16. A composition comprising:
a. a probe which can indicate the presence of a target nucleic acid, the probe comprising a nucleic acid sequence and a fluorescent metal nanocluster associated with the nucleic acid sequence; and
b. a buffer, wherein said buffer stabilizes the fluorescent metal nanocluster and associated nucleic acid sequence.

17. The composition of claim 16, wherein the buffer further comprises phosphate and/or acetate in an amount sufficient to stabilize the fluorescent metal nanocluster and associated nucleic acid sequence.

18. (canceled)

19. The composition of claim 16, wherein the probe comprises a nuclease recognition site.

20. (canceled)

21. The composition of claim 16, wherein the nucleic acid sequence of the probe comprises secondary structure.

22-31. (canceled)

32. A probe which can indicate the presence of a target nucleic acid, the probe comprising: a nucleic acid sequence, wherein said nucleic acid sequence comprises a nuclease recognition site, and a fluorescent metal nanocluster associated with the nucleic acid sequence.

33-96. (canceled)

97. A method of detecting a target nucleic acid in a sample, the method comprising:
a. contacting the sample with:
i. at least one ribonucleoprotein (RNP) comprising a guide molecule specific for at least one target nucleic acid and an effector molecule which is capable of cleaving a probe upon binding of the guide molecule in the presence of target nucleic acids; and
ii. at least one probe comprising a nucleic acid sequence and a fluorescent metal nanocluster associated with the nucleic acid sequence; and
b. measuring a detectable signal produced by cleavage of the probe by the effector molecule upon binding of the guide molecule, thereby detecting the target nucleic acid.

98-131. (canceled)


SEQUENCE LISTING (112 sequences)

SEQ ID NO: 1 - DNA, 40 bp, synthetic construct
  misc_feature 1..40: note = Description of sequence: Probe
  source 1..40: mol_type = other DNA
ggccctgcgatttttttttttttttttttttttttttttt

SEQ ID NO: 2 - DNA, 26 bp, synthetic construct
  misc_feature 1..26: note = Description of sequence: Probe
  source 1..26: mol_type = other DNA
gggttagggtccccccacccttaccc

SEQ ID NO: 3 - DNA, 30 bp, synthetic construct
  misc_feature 1..30: note = Description of sequence: Probe
  source 1..30: mol_type = other DNA
aatccccccatttttttttttttttttttt

SEQ ID NO: 4 - DNA, 10 bp, synthetic construct
  misc_feature 1..10: note = Description of sequence: Probe
  source 1..10: mol_type = other DNA
aatcccccca

SEQ ID NO: 5 - DNA, 26 bp, synthetic construct
  misc_feature 1..26: note = Description of sequence: Probe
  source 1..26: mol_type = other DNA
gggttagggtccccccacccttaccc

SEQ ID NO: 6 - DNA, 44 bp, synthetic construct
  misc_feature 1..44: note = Description of sequence: Probe
  source 1..44: mol_type = other DNA
tccccaacaattgtccgacctgcagtgatgacaaaatccccaac

SEQ ID NO: 7 - DNA, 55 bp, synthetic construct
  misc_feature 1..55: note = Description of sequence: Probe
  source 1..55: mol_type = other DNA
cccttaatccccaattgtccgacctgcagtgatgacaaaaccccctaattccccc

SEQ ID NO: 8 - DNA, 10 bp, synthetic construct
  misc_feature 1..10: note = Description of sequence: Probe
  source 1..10: mol_type = other DNA
gatccccaac

SEQ ID NO: 9 - DNA, 37 bp, synthetic construct
  misc_feature 1..37: note = Description of sequence: Probe
  source 1..37: mol_type = other DNA
aaccaccccattgtcttttttaagacaagttcccccc

SEQ ID NO: 10 - DNA, 37 bp, synthetic construct
  misc_feature 1..37: note = Description of sequence: Probe
  source 1..37: mol_type = other DNA
aaccaccccatcgtcttttttaagacgagttcccccc

SEQ ID NO: 11 - DNA, 37 bp, synthetic construct
  misc_feature 1..37: note = Description of sequence: Probe
  source 1..37: mol_type = other DNA
caccaccccattgtcttttttaagacaagttcccccc

SEQ ID NO: 12 - DNA, 37 bp, synthetic construct
  misc_feature 1..37: note = Description of sequence: Probe
  source 1..37: mol_type = other DNA
gaccaccccattgtcttttttaagacaagttcccccc

SEQ ID NO: 13 - DNA, 37 bp, synthetic construct
  misc_feature 1..37: note = Description of sequence: Probe
  source 1..37: mol_type = other DNA
taccaccccattgtcttttttaagacaagttcccccc

SEQ ID NO: 14 - DNA, 36 bp, synthetic construct
  misc_feature 1..36: note = Description of sequence: Probe
  source 1..36: mol_type = other DNA
accaccccattgtcttttttaagacaagttcccccc

SEQ ID NO: 15 - DNA, 37 bp, synthetic construct
  misc_feature 1..37: note = Description of sequence: Probe
  source 1..37: mol_type = other DNA
acccaccccattgtcttttttaagacaagttcccccc

SEQ ID NO: 16 - DNA, 37 bp, synthetic construct
  misc_feature 1..37: note = Description of sequence: Probe
  source 1..37: mol_type = other DNA
agccaccccattgtcttttttaagacaagttcccccc

SEQ ID NO: 17 - DNA, 37 bp, synthetic construct
  misc_feature 1..37: note = Description of sequence: Probe
  source 1..37: mol_type = other DNA
atccaccccattgtcttttttaagacaagttcccccc

SEQ ID NO: 18 - DNA, 36 bp, synthetic construct; identical to SEQ ID NO: 14
  misc_feature 1..36: note = Description of sequence: Probe
  source 1..36: mol_type = other DNA

SEQ ID NO: 19 - DNA, 37 bp, synthetic construct
  misc_feature 1..37: note = Description of sequence: Probe
  source 1..37: mol_type = other DNA
aaacaccccattgtcttttttaagacaagttcccccc

SEQ ID NO: 20 - DNA, 37 bp, synthetic construct
  misc_feature 1..37: note = Description of sequence: Probe
  source 1..37: mol_type = other DNA
aagcaccccattgtcttttttaagacaagttcccccc

SEQ ID NO: 21 - DNA, 37 bp, synthetic construct
  misc_feature 1..37: note = Description of sequence: Probe
  source 1..37: mol_type = other DNA
aatcaccccattgtcttttttaagacaagttcccccc

SEQ ID NO: 22 - DNA, 36 bp, synthetic construct
  misc_feature 1..36: note = Description of sequence: Probe
  source 1..36: mol_type = other DNA
aacaccccattgtcttttttaagacaagttcccccc

SEQ ID NO: 23 - DNA, 37 bp, synthetic construct
  misc_feature 1..37: note = Description of sequence: Probe
  source 1..37: mol_type = other DNA
aacaaccccattgtcttttttaagacaagttcccccc

SEQ ID NO: 24 - DNA, 37 bp, synthetic construct
  misc_feature 1..37: note = Description of sequence: Probe
  source 1..37: mol_type = other DNA
aacgaccccattgtcttttttaagacaagttcccccc

SEQ ID NO: 25 - DNA, 37 bp, synthetic construct
  misc_feature 1..37: note = Description of sequence: Probe
  source 1..37: mol_type = other DNA
aactaccccattgtcttttttaagacaagttcccccc

SEQ ID NO: 26 - DNA, 36 bp, synthetic construct; identical to SEQ ID NO: 22
  misc_feature 1..36: note = Description of sequence: Probe
  source 1..36: mol_type = other DNA

SEQ ID NO: 27 - DNA, 37 bp, synthetic construct
  misc_feature 1..37: note = Description of sequence: Probe
  source 1..37: mol_type = other DNA
aacccccccattgtcttttttaagacaagttcccccc

SEQ ID NO: 28 - DNA, 37 bp, synthetic construct
  misc_feature 1..37: note = Description of sequence: Probe
  source 1..37: mol_type = other DNA
aaccgccccattgtcttttttaagacaagttcccccc

SEQ ID NO: 29 - DNA, 37 bp, synthetic construct
  misc_feature 1..37: note = Description of sequence: Probe
  source 1..37: mol_type = other DNA
aacctccccattgtcttttttaagacaagttcccccc

SEQ ID NO: 30 - DNA, 36 bp, synthetic construct
  misc_feature 1..36: note = Description of sequence: Probe
  source 1..36: mol_type = other DNA
aaccccccattgtcttttttaagacaagttcccccc

SEQ ID NO: 31 - DNA, 37 bp, synthetic construct
  misc_feature 1..37: note = Description of sequence: Probe
  source 1..37: mol_type = other DNA
aaccaacccattgtcttttttaagacaagttcccccc

SEQ ID NO: 32 - DNA, 37 bp, synthetic construct
  misc_feature 1..37: note = Description of sequence: Probe
  source 1..37: mol_type = other DNA
aaccagcccattgtcttttttaagacaagttcccccc

SEQ ID NO: 33 - DNA, 37 bp, synthetic construct
  misc_feature 1..37: note = Description of sequence: Probe
  source 1..37: mol_type = other DNA
aaccatcccattgtcttttttaagacaagttcccccc

SEQ ID NO: 34 - DNA, 36 bp, synthetic construct
  misc_feature 1..36: note = Description of sequence: Probe
  source 1..36: mol_type = other DNA
aaccacccattgtcttttttaagacaagttcccccc

SEQ ID NO: 35 - DNA, 37 bp, synthetic construct
  misc_feature 1..37: note = Description of sequence: Probe
  source 1..37: mol_type = other DNA
aaccacaccattgtcttttttaagacaagttcccccc

SEQ ID NO: 36 - DNA, 37 bp, synthetic construct
  misc_feature 1..37: note = Description of sequence: Probe
  source 1..37: mol_type = other DNA
aaccacgccattgtcttttttaagacaagttcccccc

SEQ ID NO: 37 - DNA, 37 bp, synthetic construct
  misc_feature 1..37: note = Description of sequence: Probe
  source 1..37: mol_type = other DNA
aaccactccattgtcttttttaagacaagttcccccc

SEQ ID NO: 38 - DNA, 36 bp, synthetic construct; identical to SEQ ID NO: 34
  misc_feature 1..36: note = Description of sequence: Probe
  source 1..36: mol_type = other DNA

SEQ ID NO: 39 - DNA, 37 bp, synthetic construct
  misc_feature 1..37: note = Description of sequence: Probe
  source 1..37: mol_type = other DNA
aaccaccacattgtcttttttaagacaagttcccccc

SEQ ID NO: 40 - DNA, 37 bp, synthetic construct
  misc_feature 1..37: note = Description of sequence: Probe
  source 1..37: mol_type = other DNA
aaccaccgcattgtcttttttaagacaagttcccccc

SEQ ID NO: 41 - DNA, 37 bp, synthetic construct
  misc_feature 1..37: note = Description of sequence: Probe
  source 1..37: mol_type = other DNA
aaccacctcattgtcttttttaagacaagttcccccc

SEQ ID NO: 42 - DNA, 36 bp, synthetic construct; identical to SEQ ID NO: 34
  misc_feature 1..36: note = Description of sequence: Probe
  source 1..36: mol_type = other DNA

SEQ ID NO: 43 - DNA, 37 bp, synthetic construct
  misc_feature 1..37: note = Description of sequence: Probe
  source 1..37: mol_type = other DNA
aaccacccaattgtcttttttaagacaagttcccccc

SEQ ID NO: 44 - DNA, 37 bp, synthetic construct
  misc_feature 1..37: note = Description of sequence: Probe
  source 1..37: mol_type = other DNA
aaccacccgattgtcttttttaagacaagttcccccc

SEQ ID NO: 45 - DNA, 37 bp, synthetic construct
  misc_feature 1..37: note = Description of sequence: Probe
  source 1..37: mol_type = other DNA
aaccaccctattgtcttttttaagacaagttcccccc

SEQ ID NO: 46 - DNA, 36 bp, synthetic construct; identical to SEQ ID NO: 34
  misc_feature 1..36: note = Description of sequence: Probe
  source 1..36: mol_type = other DNA

SEQ ID NO: 47 - DNA, 37 bp, synthetic construct
  misc_feature 1..37: note = Description of sequence: Probe
  source 1..37: mol_type = other DNA
aaccacccccttgtcttttttaagacaagttcccccc

SEQ ID NO: 48 - DNA, 37 bp, synthetic construct
  misc_feature 1..37: note = Description of sequence: Probe
  source 1..37: mol_type = other DNA
aaccaccccgttgtcttttttaagacaagttcccccc

SEQ ID NO: 49 - DNA, 37 bp, synthetic construct
  misc_feature 1..37: note = Description of sequence: Probe
  source 1..37: mol_type = other DNA
aaccacccctttgtcttttttaagacaagttcccccc

SEQ ID NO: 50 - DNA, 37 bp, synthetic construct
  misc_feature 1..37: note = Description of sequence: Probe
  source 1..37: mol_type = other DNA
aaccaccccaatgtcttttttaagacatgttcccccc

SEQ ID NO: 51 - DNA, 37 bp, synthetic construct
  misc_feature 1..37: note = Description of sequence: Probe
  source 1..37: mol_type = other DNA
aaccaccccactgtcttttttaagacaggttcccccc

SEQ ID NO: 52 - DNA, 37 bp, synthetic construct
  misc_feature 1..37: note = Description of sequence: Probe
  source 1..37: mol_type = other DNA
aaccaccccagtgtcttttttaagacacgttcccccc

SEQ ID NO: 53 - DNA, 35 bp, synthetic construct
  misc_feature 1..35: note = Description of sequence: Probe
  source 1..35: mol_type = other DNA
aaccaccccatgtcttttttaagacagttcccccc

SEQ ID NO: 54 - DNA, 37 bp, synthetic construct
  misc_feature 1..37: note = Description of sequence: Probe
  source 1..37: mol_type = other DNA
aaccaccccatagtcttttttaagactagttcccccc

SEQ ID NO: 55 - DNA, 37 bp, synthetic construct; identical to SEQ ID NO: 10
  misc_feature 1..37: note = Description of sequence: Probe
  source 1..37: mol_type = other DNA

SEQ ID NO: 56 - DNA, 35 bp, synthetic construct; identical to SEQ ID NO: 53
  misc_feature 1..35: note = Description of sequence: Probe
  source 1..35: mol_type = other DNA

SEQ ID NO: 57 - DNA, 37 bp, synthetic construct
  misc_feature 1..37: note = Description of sequence: Probe
  source 1..37: mol_type = other DNA
aaccaccccattatcttttttaagataagttcccccc

SEQ ID NO: 58 - DNA, 37 bp, synthetic construct
  misc_feature 1..37: note = Description of sequence: Probe
  source 1..37: mol_type = other DNA
aaccaccccattctcttttttaagagaagttcccccc

SEQ ID NO: 59 - DNA, 37 bp, synthetic construct
  misc_feature 1..37: note = Description of sequence: Probe
  source 1..37: mol_type = other DNA
aaccaccccattttcttttttaagaaaagttcccccc

SEQ ID NO: 60 - DNA, 35 bp, synthetic construct
  misc_feature 1..35: note = Description of sequence: Probe
  source 1..35: mol_type = other DNA
aaccaccccatttcttttttaagaaagttcccccc

SEQ ID NO: 61 - DNA, 37 bp, synthetic construct
  misc_feature 1..37: note = Description of sequence: Probe
  source 1..37: mol_type = other DNA
aaccaccccattgacttttttaagtcaagttcccccc

SEQ ID NO: 62 - DNA, 37 bp, synthetic construct
  misc_feature 1..37: note = Description of sequence: Probe
  source 1..37: mol_type = other DNA
aaccaccccattgccttttttaaggcaagttcccccc

SEQ ID NO: 63 - DNA, 37 bp, synthetic construct
  misc_feature 1..37: note = Description of sequence: Probe
  source 1..37: mol_type = other DNA
aaccaccccattggcttttttaagccaagttcccccc

SEQ ID NO: 64 - DNA, 35 bp, synthetic construct
  misc_feature 1..35: note = Description of sequence: Probe
  source 1..35: mol_type = other DNA
aaccaccccattgcttttttaagcaagttcccccc

SEQ ID NO: 65 - DNA, 37 bp, synthetic construct
  misc_feature 1..37: note = Description of sequence: Probe
  source 1..37: mol_type = other DNA
aaccaccccattgtattttttaatacaagttcccccc

SEQ ID NO: 66 - DNA, 37 bp, synthetic construct
  misc_feature 1..37: note = Description of sequence: Probe
  source 1..37: mol_type = other DNA
aaccaccccattgtgttttttaacacaagttcccccc

SEQ ID NO: 67 - DNA, 37 bp, synthetic construct
  misc_feature 1..37: note = Description of sequence: Probe
  source 1..37: mol_type = other DNA
aaccaccccattgttttttttaaaacaagttcccccc

SEQ ID NO: 68 - DNA, 35 bp, synthetic construct
  misc_feature 1..35: note = Description of sequence: Probe
  source 1..35: mol_type = other DNA
aaccaccccattgtttttttaaacaagttcccccc

SEQ ID NO: 69 - DNA, 37 bp, synthetic construct
  misc_feature 1..37: note = Description of sequence: Probe
  source 1..37: mol_type = other DNA
aaccaccccattgtcatttttatgacaagttcccccc

SEQ ID NO: 70 - DNA, 37 bp, synthetic construct
  misc_feature 1..37: note = Description of sequence: Probe
  source 1..37: mol_type = other DNA
aaccaccccattgtcctttttaggacaagttcccccc

SEQ ID NO: 71 - DNA, 37 bp, synthetic construct
  misc_feature 1..37: note = Description of sequence: Probe
  source 1..37: mol_type = other DNA
aaccaccccattgtcgtttttacgacaagttcccccc

SEQ ID NO: 72 - DNA, 35 bp, synthetic construct
  misc_feature 1..35: note = Description of sequence: Probe
  source 1..35: mol_type = other DNA
aaccaccccattgtctttttagacaagttcccccc

SEQ ID NO: 73 - DNA, 37 bp, synthetic construct
  misc_feature 1..37: note = Description of sequence: Probe
  source 1..37: mol_type = other DNA
aaccaccccattgtctatttttagacaagttcccccc

SEQ ID NO: 74 - DNA, 37 bp, synthetic construct
  misc_feature 1..37: note = Description of sequence: Probe
  source 1..37: mol_type = other DNA
aaccaccccattgtctcttttgagacaagttcccccc

SEQ ID NO: 75 - DNA, 37 bp, synthetic construct
  misc_feature 1..37: note = Description of sequence: Probe
  source 1..37: mol_type = other DNA
aaccaccccattgtctgttttcagacaagttcccccc

SEQ ID NO: 76 - DNA, 35 bp, synthetic construct; identical to SEQ ID NO: 72
  misc_feature 1..35: note = Description of sequence: Probe
  source 1..35: mol_type = other DNA

SEQ ID NO: 77 - DNA, 37 bp, synthetic construct
  misc_feature 1..37: note = Description of sequence: Probe
  source 1..37: mol_type = other DNA
aaccaccccattgtcttttttaagacaaattcccccc

SEQ ID NO: 78 - DNA, 37 bp, synthetic construct
  misc_feature 1..37: note = Description of sequence: Probe
  source 1..37: mol_type = other DNA
aaccaccccattgtcttttttaagacaacttcccccc

SEQ ID NO: 79 - DNA, 37 bp, synthetic construct
  misc_feature 1..37: note = Description of sequence: Probe
  source 1..37: mol_type = other DNA
aaccaccccattgtcttttttaagacaatttcccccc

SEQ ID NO: 80 - DNA, 36 bp, synthetic construct
  misc_feature 1..36: note = Description of sequence: Probe
  source 1..36: mol_type = other DNA
aaccaccccattgtcttttttaagacaattcccccc

SEQ ID NO: 81 - DNA, 37 bp, synthetic construct
  misc_feature 1..37: note = Description of sequence: Probe
  source 1..37: mol_type = other DNA
aaccaccccattgtcttttttaagacaagatcccccc

SEQ ID NO: 82 - DNA, 37 bp, synthetic construct
  misc_feature 1..37: note = Description of sequence: Probe
  source 1..37: mol_type = other DNA
aaccaccccattgtcttttttaagacaagctcccccc

SEQ ID NO: 83 - DNA, 37 bp, synthetic construct
  misc_feature 1..37: note = Description of sequence: Probe
  source 1..37: mol_type = other DNA
aaccaccccattgtcttttttaagacaaggtcccccc

SEQ ID NO: 84 - DNA, 36 bp, synthetic construct
  misc_feature 1..36: note = Description of sequence: Probe
  source 1..36: mol_type = other DNA
aaccaccccattgtcttttttaagacaagtcccccc

SEQ ID NO: 85 - DNA, 37 bp, synthetic construct
  misc_feature 1..37: note = Description of sequence: Probe
  source 1..37: mol_type = other DNA
aaccaccccattgtcttttttaagacaagtacccccc

SEQ ID NO: 86 - DNA, 37 bp, synthetic construct
  misc_feature 1..37: note = Description of sequence: Probe
  source 1..37: mol_type = other DNA
aaccaccccattgtcttttttaagacaagtccccccc

SEQ ID NO: 87 - DNA, 37 bp, synthetic construct
  misc_feature 1..37: note = Description of sequence: Probe
  source 1..37: mol_type = other DNA
aaccaccccattgtcttttttaagacaagtgcccccc

SEQ ID NO: 88 - DNA, 36 bp, synthetic construct; identical to SEQ ID NO: 84
  misc_feature 1..36: note = Description of sequence: Probe
  source 1..36: mol_type = other DNA

SEQ ID NO: 89 - DNA, 37 bp, synthetic construct
  misc_feature 1..37: note = Description of sequence: Probe
  source 1..37: mol_type = other DNA
aaccaccccattgtcttttttaagacaagttaccccc

SEQ ID NO: 90 - DNA, 36 bp, synthetic construct
  misc_feature 1..36: note = Description of sequence: Probe
  source 1..36: mol_type = other DNA
aaccaccccattgtcttttttaagacaagttacccc

SEQ ID NO: 91 - DNA, 37 bp, synthetic construct
  misc_feature 1..37: note = Description of sequence: Probe
  source 1..37: mol_type = other DNA
aaccaccccattgtcttttttaagacaagttccaccc

SEQ ID NO: 92 - DNA, 37 bp, synthetic construct
  misc_feature 1..37: note = Description of sequence: Probe
  source 1..37: mol_type = other DNA
aaccaccccattgtcttttttaagacaagttccgccc

SEQ ID NO: 93 - DNA, 37 bp, synthetic construct
  misc_feature 1..37: note = Description of sequence: Probe
  source 1..37: mol_type = other DNA
aaccaccccattgtcttttttaagacaagttcctccc

SEQ ID NO: 94 - DNA, 36 bp, synthetic construct
  misc_feature 1..36: note = Description of sequence: Probe
  source 1..36: mol_type = other DNA
aaccaccccattgtcttttttaagacaagttccccc

SEQ ID NO: 95 - DNA, 37 bp, synthetic construct
  misc_feature 1..37: note = Description of sequence: Probe
  source 1..37: mol_type = other DNA
aaccaccccattgtcttttttaagacaagttcccacc

SEQ ID NO: 96 - DNA, 37 bp, synthetic construct
  misc_feature 1..37: note = Description of sequence: Probe
  source 1..37: mol_type = other DNA
aaccaccccattgtcttttttaagacaagttcccgcc

SEQ ID NO: 97 - DNA, 37 bp, synthetic construct
  misc_feature 1..37: note = Description of sequence: Probe
  source 1..37: mol_type = other DNA
aaccaccccattgtcttttttaagacaagttccctcc

SEQ ID NO: 98 - DNA, 36 bp, synthetic construct; identical to SEQ ID NO: 94
  misc_feature 1..36: note = Description of sequence: Probe
  source 1..36: mol_type = other DNA

SEQ ID NO: 99 - DNA, 37 bp, synthetic construct
  misc_feature 1..37: note = Description of sequence: Probe
  source 1..37: mol_type = other DNA
aaccaccccattgtcttttttaagacaagttccccac

SEQ ID NO: 100 - DNA, 37 bp, synthetic construct
  misc_feature 1..37: note = Description of sequence: Probe
  source 1..37: mol_type = other DNA
aaccaccccattgtcttttttaagacaagttccccgc

SEQ ID NO: 101 - DNA, 37 bp, synthetic construct
  misc_feature 1..37: note = Description of sequence: Probe
  source 1..37: mol_type = other DNA
aaccaccccattgtcttttttaagacaagttcccctc

SEQ ID NO: 102 - DNA, 36 bp, synthetic construct; identical to SEQ ID NO: 94
  misc_feature 1..36: note = Description of sequence: Probe
  source 1..36: mol_type = other DNA

SEQ ID NO: 103 - DNA, 37 bp, synthetic construct
  misc_feature 1..37: note = Description of sequence: Probe
  source 1..37: mol_type = other DNA
aaccaccccattgtcttttttaagacaagttccccca

SEQ ID NO: 104 - DNA, 37 bp, synthetic construct
  misc_feature 1..37: note = Description of sequence: Probe
  source 1..37: mol_type = other DNA
aaccaccccattgtcttttttaagacaagttcccccg

SEQ ID NO: 105 - DNA, 37 bp, synthetic construct
  misc_feature 1..37: note = Description of sequence: Probe
  source 1..37: mol_type = other DNA
aaccaccccattgtcttttttaagacaagttccccct

SEQ ID NO: 106 - DNA, 36 bp, synthetic construct; identical to SEQ ID NO: 94
  misc_feature 1..36: note = Description of sequence: Probe
  source 1..36: mol_type = other DNA

SEQ ID NO: 107 - DNA, 37 bp, synthetic construct; identical to SEQ ID NO: 89
  misc_feature 1..37: note = Description of sequence: Probe
  source 1..37: mol_type = other DNA

SEQ ID NO: 108 - DNA, 37 bp, synthetic construct
  misc_feature 1..37: note = Description of sequence: Probe
  source 1..37: mol_type = other DNA
aaccaccccattgtcttttttaagacaagttgccccc

SEQ ID NO: 109 - DNA, 37 bp, synthetic construct
  misc_feature 1..37: note = Description of sequence: Probe
  source 1..37: mol_type = other DNA
aaccaccccattgtcttttttaagacaagtttccccc

SEQ ID NO: 110 - DNA, 37 bp, synthetic construct
  misc_feature 1..37: note = Description of sequence: Probe
  source 1..37: mol_type = other DNA
aaccaccccattgtcttttttaagacaagttcacccc

SEQ ID NO: 111 - DNA, 36 bp, synthetic construct
  misc_feature 1..36: note = Description of sequence: Probe
  source 1..36: mol_type = other DNA
accaccccattgtcttttttaagacaagttcgcccc

SEQ ID NO: 112 - DNA, 37 bp, synthetic construct
  misc_feature 1..37: note = Description of sequence: Probe
  source 1..37: mol_type = other DNA
aaccaccccattgtcttttttaagacaagttctcccc